(19) United States
(12) Patent Application Publication
(10) Pub. No.: US 2015/0374233 A1
(43) Pub. Date: Dec. 31, 2015

(54) Visual Problem Diagnosis Using Refractive Parameters Measured With A Retinal Camera

(71) Applicant: GuoTong Medical Development Co., Ltd, Shanghai (CN)

(72) Inventors: JinJun Zhang, Shanghai (CN); Yang Yang, Shanghai (CN); John Marshall, Shanghai (CN)

(21) Appl. No.: 14/755,616

(22) Filed: Jun. 30, 2015

Related U.S. Application Data
(60) Provisional application No. 62/019,182, filed on Jun. 30, 2014.

(51) Int. Cl. A61B 3/12

(52) U.S. Cl. CPC A61B 3/12

(57) ABSTRACT

Systems, devices, and methods used to diagnose visual problems are disclosed. Diagnosing visual problems can be implemented using a fundus camera, for example. The camera focuses an image of an interesting portion of an eye being viewed. The image can be an intermediate real image of the fundus. Once the portion of the eye is in focus, the settings or the position of the focusing mechanism is determined. An optical error of an eye can be determined based on the determined settings or the determined position of the focusing mechanism. Once in focus, an image of the interesting portion of the eye can be taken or captured. A size of any feature of the eye can be determined, in absolute units based on determining an area occupied by the feature of the eye in the picture or the image, and the determined optical error of the eye.

### CROSS-REFERENCE TO RELATED APPLICATIONS/INCORPORATION BY REFERENCE

This application makes reference to and claims priority to U.S. Provisional Application Ser. No. 62/019,182 filed on Jun. 30, 2014, which is hereby incorporated herein by reference in its entirety.

### FEDERALLY SPONSORED RESEARCH OR DEVELOPMENT

[Not Applicable]

### MICROFICHE/COPYRIGHT REFERENCE

[Not Applicable]

### FIELD

Certain embodiments of the invention relate to signal processing. More specifically, certain embodiments of the invention relate to a method and system for visual problem diagnosis using refractive parameters measured with a retinal camera.

### BACKGROUND

Fundus photography or fundography is the creation of a photograph or image (e.g., a digital image) of the interior surface of the eye which can include, for example, one of more of the following: the retina, optic disc, macula, and posterior pole.

Fundus photography is used by optometrists, ophthalmologists, and trained medical professionals for monitoring progression of a disease, diagnosis of a disease (combined with retinal angiography), or in screening programs and epidemiology.

Further limitations and disadvantages of conventional and traditional approaches will become apparent to one of skill in the art, through comparison of such systems with the present invention as set forth in the remainder of the present application with reference to the drawings.

### BRIEF DESCRIPTION OF THE DRAWINGS

FIG. 1 shows different types of simple lenses.

FIG. 2 shows a lens that is biconvex or plano-convex.

FIG. 3 shows a lens the lens that is biconcave or plano-concave.

FIG. 4 shows a real image generated by a lens.

FIG. 5 shows a virtual image generated by a lens.

FIG. 6 shows a plane in which the subject is focused.

FIG. 7 shows a camera display for use in a focusing method.

FIG. 8 shows a Hartmann mask or Scheiner disk.

FIG. 9 shows a Bahtinov mask.

FIG. 10 shows knife edge focusing.

FIG. 11 shows imaging in direct ophthalmoscopy.

FIG. 12 shows a patient's illuminated fundus in a method that uses direct ophthalmoscopy.

FIG. 13 shows a limited field of view in a method that uses direct ophthalmoscopy.

FIG. 14 shows an extended field of view in a method that uses indirect ophthalmoscopy.

FIG. 15 shows a tracing of the trays from the patient's fundus to the observer's retina in a method that uses indirect ophthalmoscopy.

FIG. 16 shows a magnification of an aerial image in a method that uses indirect ophthalmoscopy.

FIG. 17 shows magnification for different lenses in a method that uses indirect ophthalmoscopy.

FIG. 18 shows distances, magnifications, and calculations for various lenses and viewing distances in a method that uses indirect ophthalmoscopy.

FIG. 19 shows compensation for refractive error in a method that uses indirect ophthalmoscopy.

FIG. 20 shows the field of view with and without a lens.

FIG. 21 shows an SLR viewing system of a fundus camera.

FIG. 22A shows a configuration of the fundus camera when the photographer is viewing the subject.

FIG. 22B shows a configuration of the fundus camera when the film is exposed to light from the subject.

FIG. 22C shows a configuration of the fundus camera when the distance between the focusing screen and lens system in the fundus camera and the distance between the film plane and the lens system are equal.

FIG. 23 shows various designs of focusing plane reticles.

FIG. 24 show a myopic three-surface eye and the application of the telecentric principle.

FIG. 25 shows a fundus camera and fundus imaging system in a focusing system.

FIG. 26 shows a flowchart illustrating an embodiment of a method for diagnosing a visual problem.

FIG. 27 shows a flowchart illustrating an embodiment of a method for determining a size or a length of a feature of an eye in absolute units.

FIG. 28 shows a block diagram of some components of an embodiment of an imaging system or imaging subsystem.

FIG. 29 shows a retinal camera refraction.

FIG. 30 illustrates a linear regression curve of spectacle refraction and focusing position.

FIG. 31 illustrates the basic principle of the autorefractor.

### DETAILED DESCRIPTION

Compared to ophthalmoscopy, fundus photography generally uses a considerably larger instrument, but can avail the image to be examined by a specialist, for example, at another location and/or time, as well as providing photo documentation for future reference. Fundus photographs generally recreate considerably larger areas of the fundus than what can be seen at any one time with handheld ophthalmoscopes.

A purpose of a retinal camera or retinal fundus imaging system, whether it includes a point-and-shoot camera, a view camera, a digital camera, a sensor, or a film camera, is to project light onto a surface that will capture an image. Some retinal cameras and retinal fundus imaging systems share some basic mechanisms. For example, focusing can be used for producing clear diagnostic images. The focusing system determines and provides the ocular refraction or refractive status of an eye which can be registered and stored in the retinal fundus imaging systems.

Some embodiments of the present disclosure relate to methods, systems, and devices for measuring refractive errors of an eye based on focusing systems and wavefront measurements used by retinal cameras and retinal fundus imaging systems.

Some embodiments relate to extracting or acquiring information, data, or parameters of ocular refraction or the refractive errors from one or more fundus images taken by a retinal camera or a retinal fundus imaging system. The retinal camera or the retinal fundus imaging system may comprise, for example, one or more of the following: a scanning laser ophthalmoscope (SLO), an adaptive optics scanning laser ophthalmoscope (AO-SLO), an optical coherence tomography (OCT), a 3D retinal imaging system, and a retinal scanner.

Some embodiments provide that, by extracting and using stored ocular refractive information from the retinal camera or the retinal fundus imaging system, fundus images and refractive status can be provided.

Some embodiments of the present disclosure relate to methods, computing software, systems, and devices for measuring or extracting or acquiring refractive errors of an eye being photographed by incorporating or installing computing software or one or more optometer mechanism(s) to the retinal camera or retinal imaging system.

Some embodiments of the present disclosure relate to methods, computing software, systems, and devices for measuring or extracting or acquiring refractive errors of an eye being photographed based on focusing systems and wavefront measurements used by retinal cameras and retinal fundus imaging systems.

Some embodiments of the present disclosure relate to methods and computing software for extracting or acquiring refractive errors of an eye being photographed by incorporating or installing computing software into retinal cameras and retinal fundus imaging systems.

Some embodiments of the present disclosure relate to methods, computing software, systems, and devices for measuring refractive errors of an eye being photographed by incorporating or installing one or more optometer mechanism(s) to the camera or retinal fundus imaging systems

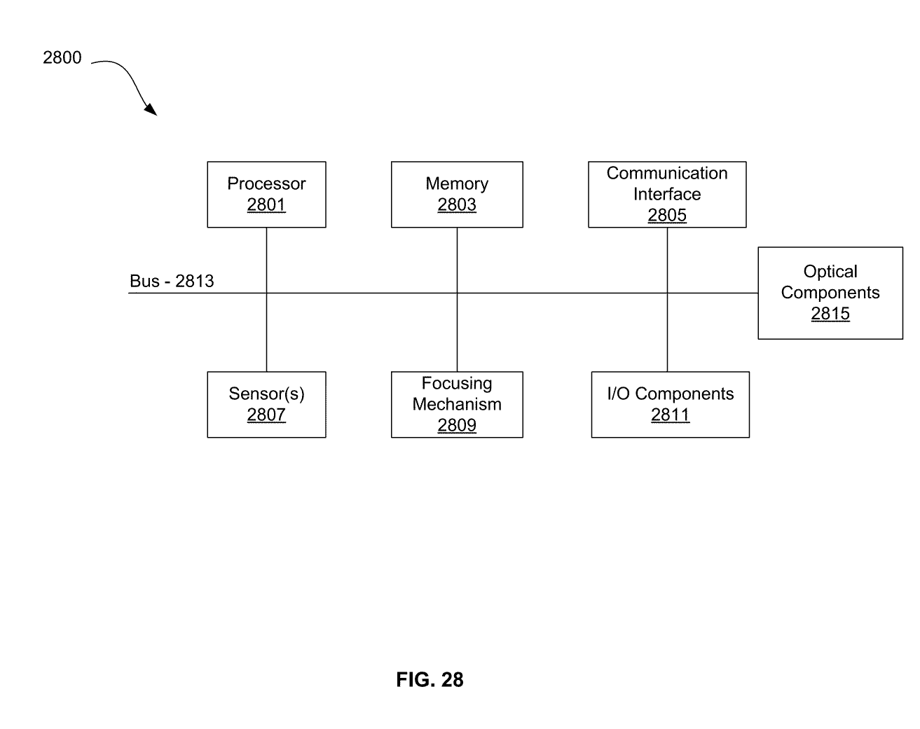

Some embodiments use one or more processors that execute instructions to perform operations or calculations based on formulas, equations and/or algorithms to determine and provide an ocular refraction of an eye. In general, executable instructions and/or code that perform operations and/or calculations described in the present application are accessible by the one or more processors and can be stored in non-transitory storage media such as, for example, one or more of the following: RAM, ROM, cache, memory, hard drive, flash drive, magnetic memory, optical memory, semiconductor memory, internal processor memory, etc. The one or more processors and the non-transitory storage media can be part of a fundus camera, a fundus imaging system, and/or a focusing system. See, e.g., FIG. 28.

Optical Principles

The optical design of a fundus camera is based on monocular indirect ophthalmoscopy. The fundus camera provides an upright, magnified view of the fundus. A typical camera views approximately 30° to approximately 50° of retinal area, with a magnification of approximately 2.5×, and allows some modification of this relationship through a zoom or auxiliary lens from approximately 15°, which provides approximately 5× magnification, to approximately 140° with a wide angle lens, which minifies the image by approximately half.

The optics of the fundus camera is similar in some ways to the optics of an indirect ophthalmoscope in that, for example, the observation and illumination systems follow dissimilar paths. Observation light is focused via a series of lenses through a doughnut-shaped aperture, which then passes through a central aperture to form an annulus, before passing through the camera objective lens and through the cornea onto the retina. The light reflected from the retina passes through the un-illuminated hole in the doughnut formed by the illumination system. As the light paths of the two systems are independent, there are minimal reflections of the light source captured in the formed image. The image forming rays continue towards the low powered telescopic eyepiece. When the button is pressed to take a picture, a mirror interrupts the path of the illumination system allow the light from the flash bulb to pass into the eye. Simultaneously, a mirror falls in front of the observation telescope, camera, imaging device, focusing system, or other optical instrument, which redirects the light onto the capturing medium, whether it is film or a digital charge-coupled device (CCD). Because of the tendency of the eye to accommodate while looking through a telescope, camera, imaging device, focusing system, or other optical instrument, it is useful that the exiting vergence is parallel in order for an in focus image to be formed on the capturing medium.

Since the instruments are complex in design and difficult to manufacture to clinical standards, only a few manufacturers exist including, for example: Topcon, Zeiss, Canon, Nidek, Kowa, CSO and CenterVue.

One or more elements and principles related to or applicable to retinal fundus imaging systems are described below.

Cameras

A purpose of a camera, whether it is a point-and-shoot camera, a view camera, a digital camera, or a film camera, is to project light onto a surface that will capture an image. The camera can have, for example, a series of lenses that focus light to create an image of a scene. In the case of a digital camera, instead of focusing the light onto a film, the camera focuses the light onto a semiconductor device or other type of device that records light electronically. A processor can then be used to analyze this electronic information and/or convert the information into digital data.

Optometers

An optometer is an Instrument for measuring the refractive state of the eye. There are two main types of optometers: subjective and objective. Subjective optometers rely upon the subject's judgment of sharpness or blurredness of a test object while objective ones contain an optical system which determines the vergence of light reflected from the subject's retina.

Electronic optometers in which all data appear digitally within a brief period of time after the operator has activated a signal can be of either type. Objective types (also called autorefractors or autorefractometers) have become very popular and several of these autorefractors are now providing both objective and subjective systems within the same instrument.

A Badal's Optometer is a simple, subjective optometer consisting of a single positive lens and a movable target. The vergence of light from the target, after refraction through the lens, depends upon the position of the target. The patient is instructed to move the target towards the lens from a position where it appears blurred until it becomes clear. That point (converted in dioptric value) represents the refraction of the patient's eye. This is a crude and inaccurate instrument, in which the measurement is marred by accommodation, variation in retinal image size with target distance, large depth of focus, nonlinearity of the scale, etc. Badal's improvement was to place the lens so that its focal point coincides with either the nodal point of the eye or the anterior focal point of the eye or the entrance pupil of the eye, thus overcoming the problems of the non-linear scale and the changing retinal image size.

Optometer of Fincham, coincidence is an objective optometer where the image of an illuminated fine line target is formed on the retina by passing through a small, peripheral portion of the pupil. The examiner views through a telescope with an optical doubling system, which splits the visual field into two. If the incident beam of light is not in focus on the retina, the reflected beam will not be along the optical axis and the two half lines will be seen out of alignment. Adjusting the dioptric value of the target in order to obtain alignment gives a measure of the ametropia.

An Infrared Optometer is an optometer that uses infrared light rather than visible light. This is done so that the target used in the optometer is invisible to the patient. Otherwise when it is altered, it tends to become a stimulus to accommodation. However, the instrument must be corrected for the chromatic aberration of the eye. Most modern optometers use infrared light. They are based on one of three principles: (1) retinoscopy, (2) Scheiner's experiment, (3) ophthalmoscopy (indirect).

A Young's optometer is a simple optometer consisting of a single positive lens and using the Scheiner's principle. The target is either a single point of light or a thread, which is moved back and forth until it is seen singly by the observer. When the target is out of focus, it is seen double and slightly blurred.

Conventional autorefractors typically measure how light rays are bent or “refracted” or by a measurement of intensity of light. These methods, however, do not achieve full refraction of the eye. Full refraction is “spherocylindrical” and in addition to sphere correction must also include cylinder and axis measurements. Conventional techniques use “foci”, which requires at least three lenses (a pupil lens and two other lenses acting as small telescopes) to look at areas (“foci”) in different areas of the eye and requires at least two detectors, and these two detectors must operate in at least two independent optical paths. Conventional stand-mounted autorefractors typically use a series of sensors and motors to acquire the eye. Another system of sensors and motors is used for fine optical alignment. Still another, third, system of optics and motors is used to control where the eye is looking and focused.

Lens

A lens is an optical device which transmits and refracts light, thereby converging or diverging the light. A simple lens includes a single optical element. A compound lens is an array of simple lenses (elements) with a common axis. The use of multiple elements allows more optical aberrations to be corrected than is possible with a single element. Lenses are typically made of glass or transparent plastic. Elements which refract electromagnetic radiation outside the visual spectrum are also called lenses. For instance, a microwave lens can be made from paraffin wax.

Some lenses are spherical lenses that have two surfaces that are parts of the surfaces of spheres, with the lens axis perpendicular to both surfaces. Each surface can be convex (e.g., bulging outwards from the lens), concave (e.g., depressed into the lens), or planar (e.g., flat). The line joining the centers of the spheres, which make up the lens surfaces, is called the axis of the lens. Typically, the lens axis passes through the physical center of the lens because of the way in which it is manufactured. Lenses may be cut or ground after manufacturing to give them a different shape or size. The lens axis might then not pass through the physical center of the lens.

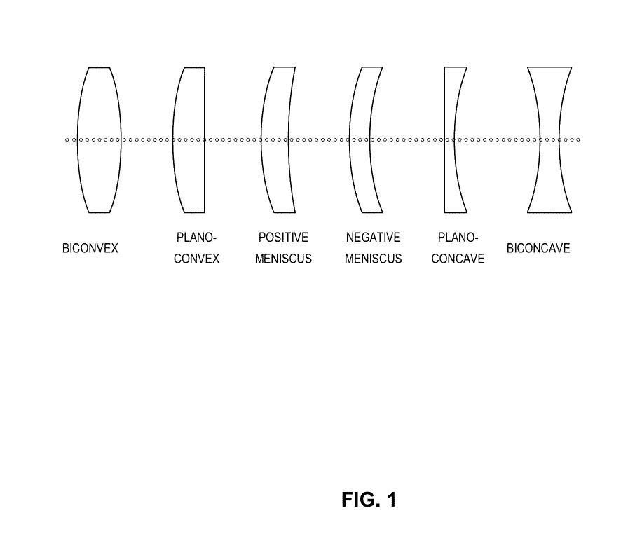

FIG. 1 shows different types of simple lenses. Lenses can be classified by the curvature of the two optical surfaces. A lens is biconvex (or double convex, or just convex) if both surfaces are convex. If both surfaces have the same radius of curvature, the lens is equi-convex. A lens with two concave surfaces is biconcave (or just concave). If one of the surfaces is flat, the lens is plano-convex or plano-concave depending on the curvature of the other surface. A lens with one convex and one concave side is convex-concave or meniscus. It is this type of lens that is commonly used in corrective lenses.

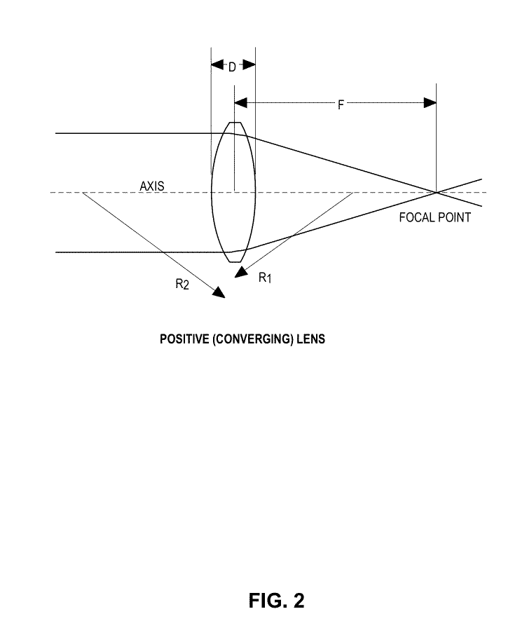

If the lens is biconvex or plano-convex as shown in FIG. 2, a collimated beam of light passing through the lens will be converged (e.g., focused) to a spot behind the lens. In this case, the lens is called a positive or converging lens. The distance from the lens to the spot is the focal length of the lens, which can be abbreviated as f in diagrams and equations.

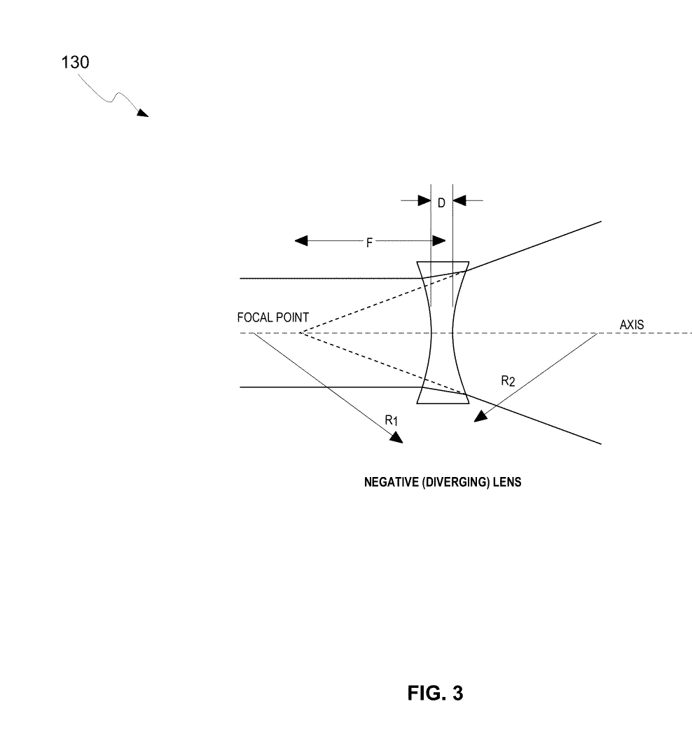

If the lens is biconcave or plano-concave as shown in FIG. 3, a collimated beam of light passing through the lens is diverged (e.g., spread). The lens is called a negative or diverging lens. The beam after passing through the lens appears to be emanating from a particular point on the axis in front of the lens. The distance from the point to the lens is also known as the focal length, although it is negative with respect to the focal length of a converging lens.

Convex-concave (e.g., meniscus) lenses can be either positive or negative, depending on the relative curvatures of the two surfaces. A negative meniscus lens has a steeper concave surface and will be thinner at the center than at the periphery. Conversely, a positive meniscus lens has a steeper convex surface and will be thicker at the center than at the periphery. An ideal thin lens with two surfaces of equal curvature has zero optical power, meaning that it would neither converge nor diverge light. All real lenses have nonzero thickness which causes a real lens with identical curved surfaces to be slightly positive. To obtain exactly zero optical power, a meniscus lens can have slightly unequal curvatures to account for the effect of the lens thickness.

Lensmaker's Equation

The focal length of a lens in air can be calculated from the lensmaker's equation:

$$P = \frac{1}{f} = ( n - 1 ) [ \frac{1}{{R}_{1}} - \frac{1}{{R}_{2}} + \frac{( n - 1 ) d}{{nR}_{1} {R}_{2}} ] ,$$

in which

P is the power of the lens,

f is the focal length of the lens,

n is the refractive index of the lens material,

R₁ is the radius of curvature (with sign, see below) of the lens surface closest to the light source,

R₂ is the radius of curvature of the lens surface farthest from the light source, and

d is the thickness of the lens (e.g., the distance along the lens axis between the two surface vertices).

The sign convention for use with the radius of curvature of a lens indicates whether the corresponding surface is convex or concave. The sign convention can vary. However, herein, if R₁ is positive the first surface is convex, and if R₁ is negative the surface is concave. The signs are reversed for the back surface of the lens: if R₂ is positive the surface is concave, and if R₂ is negative the surface is convex. If either radius is infinite, the corresponding surface is flat. With this convention the signs are determined by the shapes of the lens surfaces, and are independent of the direction in which light travels through the lens.

Imaging Properties

A positive or converging lens in air focuses a collimated beam travelling along the lens axis to a spot (known as the focal point) at a distance f from the lens. Conversely, a point source of light placed at the focal point will be converted into a collimated beam by the lens. These are two examples of image formation in lenses. In the former, an object at an infinite distance (e.g., as represented by a collimated beam of waves) is focused to an image at the focal point of the lens. In the latter, an object at the focal length distance from the lens is imaged at infinity. The plane perpendicular to the lens axis and situated at a distance f from the lens is called the focal plane.

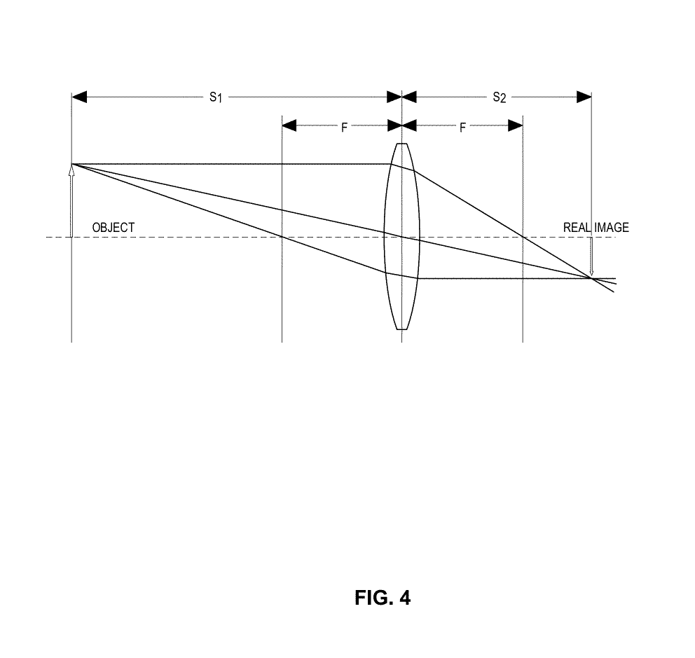

Referring to FIG. 4, if the distances from the object to the lens and from the lens to the image are S₁ and S₂, respectively, for a lens of negligible thickness, in air, the distances are related by the thin lens formula:

$$\frac{1}{{S}_{1}} + \frac{1}{{S}_{2}} = \frac{1}{f} .$$

This can also be placed into the “Newtonian” form such that

x₁x₂=f²,

in which x₁=S₁−f and x₂=S₂−f.

Thus, if an object is placed at a distance S₁ along the axis in front of a positive lens of focal length f, a screen placed at a distance S₂ behind the lens will have a sharp image of the object projected onto it, as long as S₁>f (if the lens-to-screen distance S₂ is varied slightly, the image will become less sharp). This principle is applicable to photography and the operation of the human eye. The image in this case is known as a real image.

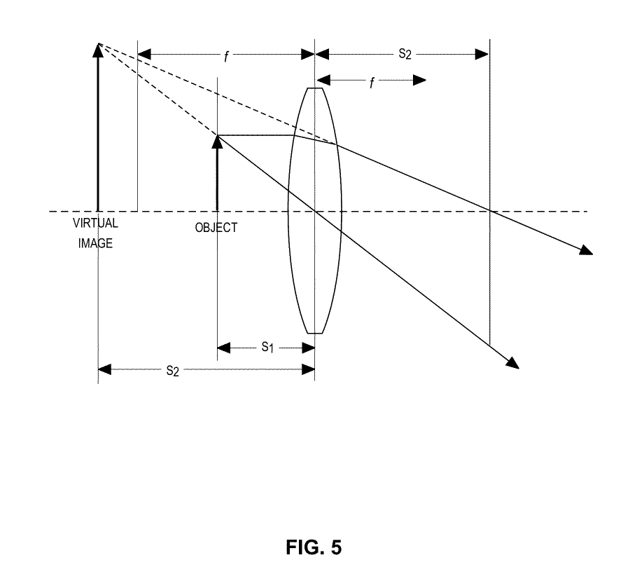

Referring to FIG. 5, if S₁<f, then S₂ becomes negative, the image is apparently positioned on the same side of the lens as the object. Although this kind of image, known as a virtual image, cannot be projected on a screen, an observer looking through the lens will see the image at its apparent calculated position. A magnifying glass creates this kind of image.

The magnification of the lens is given by:

$$M = - \frac{{S}_{2}}{{S}_{1}} = \frac{f}{f - {S}_{1}}$$

in which M is a magnification factor. If |M|>1, the image is larger than the object. According to the sign convention, if M is negative, as it is for real images, the image is upside-down with respect to the object. For virtual images, M is positive and the image is upright.

In a special case in which S₁ goes to infinity, then S₂=f and M goes to zero. This corresponds to a collimated beam being focused to a single spot at the focal point. The size of the image in this case is not actually zero, since diffraction effects place a lower limit on the size of the image (see, e.g., Rayleigh criterion).

Focusing Systems

Described herein are embodiments of focusing systems for use with a camera and/or retinal fundus imaging system, for example.

Focus is an aspect of photography that has determined the development of many different types of camera. Focus is dependent upon a number of relationships including, for example, the distance of the subject from the camera.

General photographic lenses can perform focusing by being configured for all-group focusing, in which all lens groups are moved together along the optical axis, or front-group focusing, in which only a front lens group is moved. These focusing systems can consume a lot of energy in the focus driving mechanism, thereby preventing faster auto focusing. Such focusing systems are also difficult to make the lenses compact due to the large front lens groups. The rear and inner focusing systems are developed, for example, to reduce the weight of the moving focusing lens group. The rear and inner focusing systems can employ, as a focusing lens group, optical systems other than the front lens group. Camera maker Canon uses in-house terms such as “inner focusing” meaning one or more lens groups between a front lens and a diaphragm that control focusing, and “rear focusing” meaning one or more lens groups behind the diaphragm that control focusing. In addition to speeding up autofocusing and downsizing the lens, the rear and inner focusing systems can have one or more of the following benefits: (1) easy handling of the lens because of unchanged total length of the lens during focusing; (2) easier to shorten the minimum shooting distance compared to the all-group focusing and front-group focusing methods; and (3) easier operation of polarizing filters because of non-rotating front frame.

Lens Structure

A lens bends light beams to a certain total degree, no matter the light beam's angle of entry. This total “bending angle” is determined by the structure of the lens. A lens with a rounder shape (e.g., a center that extends out farther) will have a more acute bending angle. Curving the lens out increases the distance between different points on the lens. This increases the amount of time that one part of the light wave is moving faster than another part, so the light makes a sharper turn.

Increasing the bending angle can have a number of effects. For example, light beams from a particular point will converge at a point closer to the lens. In a lens with a flatter shape, light beams will not bend as sharply. Consequently, the light beams will converge farther away from the lens. Thus, the focused real image forms farther away from the lens when the lens has a flatter surface.

Increasing the distance between the lens and the real image actually increases the total size of the real image. For example, consider a projector. As the projector is moved farther away from the screen, the image becomes larger. The light beams keep spreading apart as they travel toward the screen.

In a camera, as the distance between the lens and the real image increases, the light beams spread out more, thereby forming a larger real image, but the size of the film stays constant. When a very flat lens is attached, it projects a large real image, but the film is only exposed to the middle part of it. The lens zeroes in on the middle of the frame, magnifying a small section of the scene. A rounder lens produces a smaller real image, so the film surface sees a much wider area of the scene, for example, at a reduced magnification.

Cameras can be adapted with different lenses to view scenes at different magnifications. The magnification power of a lens is described by its focal length. In cameras, the focal length is defined as the distance between the lens and the real image of an object in the far distance (e.g., the distance to the moon). A higher focal length number indicates a greater image magnification.

A real image is formed by light moving through a convex lens. The nature of the real image varies depending on how the light travels through the lens. The light path depends on one or more of the following factors: an angle of the light beam's entry into the lens; and lens structure.

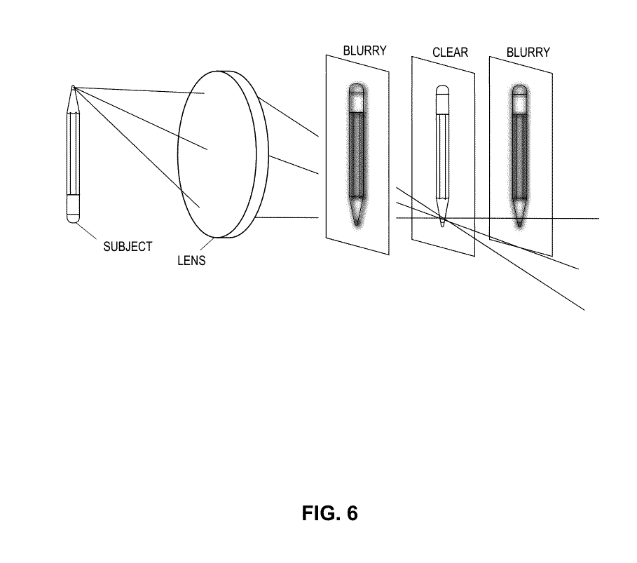

The angle of light entry changes as the object moves closer or farther away from the lens as shown in FIG. 6. The light beams from the pencil point enter the lens at a sharper angle when the pencil is closer to the lens and a more obtuse angle when the pencil is farther away. But overall, the lens only bends the light beam to a certain total degree, no matter how it enters. Consequently, light beams that enter at a sharper angle will exit at a more obtuse angle, and vice versa. The total “bending angle” at any particular point on the lens remains constant.

Light beams from a closer point converge farther away from the lens than light beams from a point that is farther away. Thus, the real image of a closer object forms farther away from the lens than the real image from a more distant object.

This phenomenon is observable with an experiment. Light a candle in the dark and position a magnifying glass between the candle and the wall. An upside down image of the candle will appear on the wall. If the real image of the candle does not fall directly on the wall, it will appear somewhat blurry since the light beams do not quite converge at this point on the wall. To focus the image, position the magnifying glass closer or farther away from the candle.

As noted above, a lens bends light beams to a certain total degree, no matter the angle of entry of the light beam. This total “bending angle” is based on the structure of the lens.

For lenses of fixed focal length (e.g., prime lenses), the usual focusing method is to move the whole lens. There are exceptions such as, for example, with high-end and low-end lenses. For cheap lenses, only one element, often the front element, can move. For some high-end lenses and zoom lenses, the focusing can be achieved by moving internal elements.

There are some high-end lenses that can combine both effects. Some high-end lenses can move the whole lens and can move internal elements (e.g., floating elements). In such high-end lenses, the internal element can be moved to improve the image quality and not to focus. For example, Nikon close range correction (CRC) lenses operate in this manner. There are some lenses, like the Zeiss 40 mm floating lens element (FLE) Distagon, in which the internal movement and focusing mechanism are separate. The floating element is set to improve image quality, and then a focusing mechanism is used to improve focus.

Macro lenses can also be focused by moving the whole lens (e.g., without moving any internal elements) and/or by moving internal elements. As well as improving the optical design for different distances, the floating elements can also reduce the focal length as the lens is focused closer. This structure is available in some Nikon macro lenses (e.g., Nikon Micro lenses), which enables the lens to focus very closely without too much lens extension. Thus, the mechanism that moves the lens further from the image plane need not have as large a displacement distance as it would have if the focal length stayed the same.

Early zoom lens designs moved the rear group (e.g., the prime group, because early zooms were like a prime lens with a variable afocal converter in front of them), but modern designs rarely move the rear group. Instead, internal elements are moved as can be seen by viewing in the front of the lens. It can be a very subtle movement.

Focusing Methods

There are many different methods of focusing. In some embodiments, it is desirable for the focusing method to be consistent and repeatable.

Focusing methods can range in accuracy from less accurate (e.g., focusing with the unaided eye on the ground glass of the camera through the pentaprism) to more accurate (e.g., examining a subject in an actual test exposure).

Many of these methods listed below can yield good results if the problems of focusing are taken into account and if the limitations and idiosyncrasies of each method are understood.

An optical system that has a slow focal ratio can be more forgiving, for example, with respect to aberrations. On the other hand, fast optical systems and high-resolution digital sensors demand critical methods for good results.

The following methods are presented, more or less, in a general order of accuracy, from least accurate to most accurate. The first five methods are generally the least accurate for truly critical focusing. Methods 6 through 13 are reasonably accurate. Methods 14 through 16 are the most accurate.

1. By Eye

2. Magnifier/Right-Angle Finder

3. Digital Zoom Trial and Error

4. Hartmann Mask or Scheiner Disk

5. Diffraction Spikes

6. Bahtinov Mask

7. Star Trail Test

8. Hybrid Method—Star Trails with a Hartmann Mask

9. Autofocus

10. In-Camera Focus Indicator

11. Parfocalized Eyepiece

12. Groundglass and Magnifier

13. Hybrid Ronchi Screen

14. Knife Edge

15. Ronchi Screen

16. Live-View Real Time Video Display

17. Software Metrics

By Eye

It would seem that the easiest way to focus is to just look through the viewfinder of the camera and to focus. It might be easy, but it is not necessarily accurate with repeatable consistency. Focusing a camera through the viewfinder with the unaided eye does not have a very high percentage of success for the vast majority of people, and even those with exceptional eyesight find it is not consistently repeatable. The accuracy of this method depends on the calibration of the reflex mirror and viewfinder system, and on user eyesight and judgment.

Magnifier/Right-Angle Finder

A better way to focus by eye is through the camera's focusing screen with the aid of a magnifier. Any additional magnification that can be used will help, but a range of approximately 15× to approximately 25× can be optimum for attempting to achieve an accurate focus. The right-angle finders make focusing possible in a more comfortable viewing position, but the dimness of the view can be a drawback. Right-angle finders are also difficult to use with any accuracy with a wide-angle lens. The accuracy of this method can depend on the calibration of the reflex mirror and viewfinder system and on user eyesight and judgment.

Digital Zoom Trial and Error

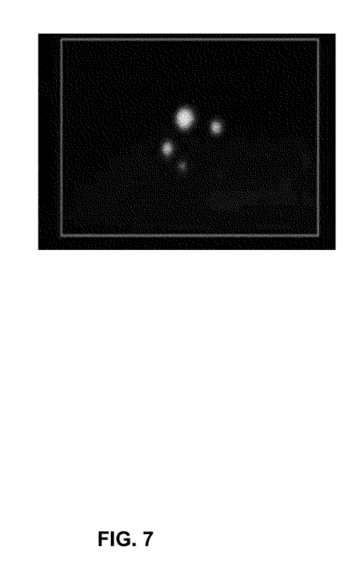

This method comprises taking short exposures and zooming in on a subject and then examining with the LCD display on the back of the camera as illustrated in FIG. 7. Then the focus can be changed slightly and the image can be examined to see if the subject looks smaller. Through a process of trial and error, the point of best focus can be determined. This method is advantageous in that it uses an actual image from the sensor for focus determination. The accuracy of this method can depend on the ability of repeatable focus positions once the correct position is determined and on user judgment as to when accurate focus is obtained.

Hartmann Mask or Scheiner Disk

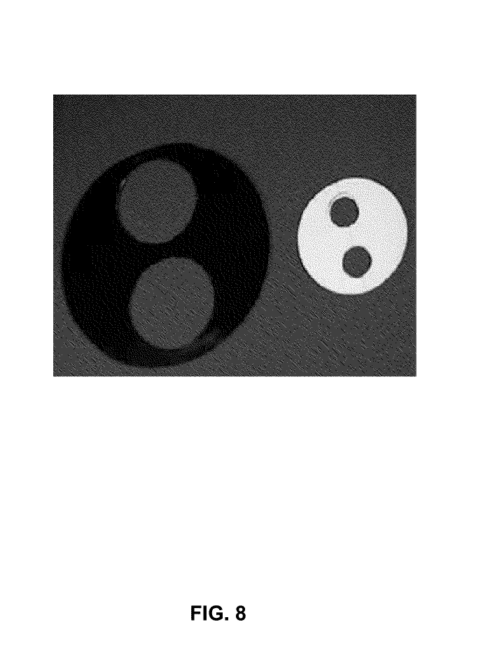

The Hartmann mask or Scheiner disk is a device comprising a set of holes in an opaque lens cover as shown in FIG. 8. The Hartmann mask can have multiple holes, while a Scheiner disk has two holes in the disk or mask. The out-of-focus images generated by each hole merge when the telescope, camera, imaging device, focusing system, or other optical instrument is in focus. They operate much in the same manner as an optical rangefinder found on rangefinder cameras, such as the Leica M series cameras. Focusing a Hartmann mask or Scheiner disk can be aided if magnification can be used on the viewfinder.

Bahtinov Mask

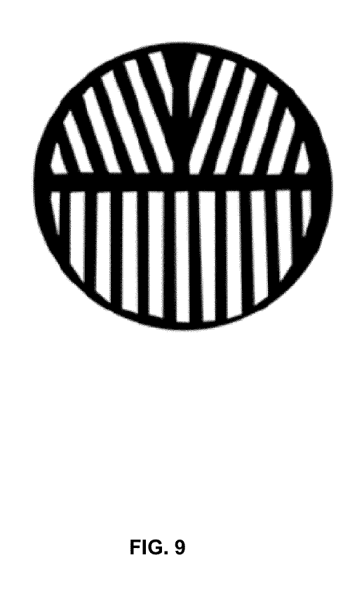

FIG. 9 shows a Bahtinov mask, which is a variation of the Hartmann mask using rectangular apertures at different angles that produce a diffraction spike pattern around the subject, with one of the diffraction spikes moving as focus is changed. When this moving spike is exactly centered in the other spikes, the scope is in focus. It is probably better to judge this on an actual test image and displayed on the camera's LCD screen. This uses the image directly from the sensor and eliminates possible errors in the camera's reflex mirror system and viewfinder display. Astrojargon has a web page that generates Bahtinov masks.

The accuracy of this system depends on the camera's viewfinder display and mirror being correctly calibrated to the imaging sensor (for visual through the viewfinder) and user judgment in evaluating the diffraction pattern to determine correct focus.

Autofocus

Most DSLR camera systems have auto-focus mechanisms built into the camera body that work in conjunction with auto-focus lenses and are sensitive enough to focus on a subject with a sufficiently fast optical system. The body and lens combinations can be used to auto-focus on a bright subject or object with sufficient contrast, but should be tested first for reliability.

Some of these systems also provide a focus indicator that works when attached to other optical systems such as telescopes, camera, imaging device, focusing system, or other optical instrument if the f/ratio speed of the optics is bright enough for the auto-focus detection system in the camera body. It has been reported that the Nikon system can work with f/ratios as slow as f/6. Nevertheless, tests should be undertaken to determine the reliability of such a method with the particular equipment.

It can be difficult to correctly place the subject exactly on the auto-focus detector because the detector is relatively small. Further, although usually well-marked on the ground glass, it can be very difficult to see against a black background in dark conditions. Shining a red flashlight down the tube of the telescope, camera, imaging device, focusing system, or other optical instrument will illuminate the ground glass and the subject can then be correctly positioned on the auto-focus detector. Once correctly placed, the flashlight should be turned off so as to not compromise the auto-focus system which works on contrast detection. Some camera bodies like the Nikon F5 illuminate the focusing rectangle that is active when the shutter button is partially depressed.

A limitation of autofocus is that it will only work on autofocus lenses, and not, for example, with a telescope, camera, imaging device, focusing system, or other optical instrument. The accuracy of this method depends on the camera's autofocus system and the sensors of the system being at the same distance as the actual focal plane of the camera's sensor.

Parfocalized Eyepiece

It is possible to parfocalize an eyepiece with the focal plane of the camera. The problem is making the eyepiece parfocalized with the sensor of the camera. To do this, the camera is first focused exactly. This can be done with another more accurate method, such as software metric-assisted focusing, knife edge, trial and error, etc. Then the eyepiece is focused by sliding it in and out of the focusing tube and locked with a parfocalizing lock-down ring. The next time the telescope, camera, imaging device, focusing system, or other optical instrument is used for this technique, it is focused visually with the parfocalized eyepiece and the camera is then substituted.

The accommodation by the eye can be an issue. The eye can make up for tiny differences in the true focus so that the image will not be exactly focused. If this method is used, the shortest focal length eyepiece possible (e.g., approximately 3 mm to approximately 5 mm) should be used to overcome the eye's accommodation. The accuracy of this method depends on the accuracy of the camera being focused first and the exact parfocalization of the eyepiece, and user eyesight and judgment.

Ground Glass and Magnifier

In this method, the ground glass may be at the same distance as the sensor in a camera. Since the image is focused on the ground glass, problems with the eye's accommodation, such as in a parfocalized eyepiece, can be avoided. Stellar Technologies makes a focuser called the CVF that takes the place of the camera and uses a ground glass and high-power magnifier to focus.

This method can also work with extended objects, which is not the case for a knife edge or Ronchi Screen. The accuracy of this method depends on the ground glass being at exactly the same distance as the camera's sensor, the magnifier being focused correctly, and user eyesight and judgment.

Hybrid Ronchi Screen

Stellar Technologies makes a Ronchi screen focuser that uses a Ronchi screen and magnifier eyepiece in a hybrid focuser called the “Stiletto.” The Stiletto takes the place of the camera for focusing. It has a right-angle diagonal which makes focusing comfortable for when a refractor or SCT is pointed at difficult angles.

Stellar Technologies also offers different screens for the Stiletto, including a standard knife-edge. The accuracy of this method depends on the size of the Airy disk that is formed by the telescope, camera, imaging device, focusing system, or other optical instrument and the size of the grating in the Ronchi screen. The accuracy of this method depends on the Ronchi screen being the exact same distance as the camera's sensor, and user eyesight and judgment.

Knife Edge

Knife edge focusing is a relatively modest concept that works remarkably well. It is not dependent on excellent eyesight and can be accomplished with or without glasses. However, it is a technique that is not easy for a beginner.

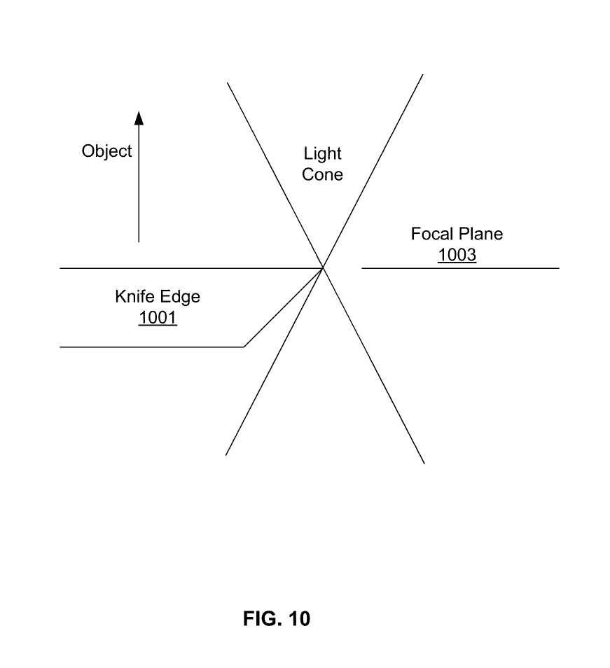

FIG. 10 illustrates knife edge focusing. A knife edge is placed in the exact same position that the film's emulsion occupies in the camera. The eye is placed slightly behind the knife edge and the image of a bright subject is examined. A point source is used to knife-edge focus. There are commercial solutions for knife-edge focusing. Hutech sells a knife edge focuser made by Mitsubishi. If the knife's edge is calibrated to the focal plane of the camera, this can be an easy and convenient way to focus because no computer is required in the field. Stellar Technologies International also makes knife edge focusers for its Stiletto series.

The accuracy of the knife-edge focusing method depends on the knife edge being at the exact same distance as the focal plane of the camera's sensor, and user eyesight and judgment.

Ronchi Screen

Focusing by a Ronchi screen is similar to knife-edge focusing. Instead of a single knife edge, a Ronchi screen has multiple lines, each of which can act as a knife edge. This makes focusing a bit easier because the subject does not have to be positioned exactly to a single edge since there are many from which to choose. Focus is indicated as the shadow of the lines become larger and less lines are visible. Very close to focus, only a single line is visible and this is the one that acts as the knife edge to finalize focus.

As with a knife-edge focuser, the accuracy of the screen depends on the Ronchi lines being at the exact same distance as the focal plane of the camera sensor, and the user judgment in evaluating the position of correct focus.

Live-View Real Time Video Display

Video from the sensor in the camera can be displayed on the LCD on the back of the camera in real time. This is a feature that is available on almost every point-and-shoot digital snapshot camera, but has not been available in the past on most DSLR cameras except the Fuji FinePix S3Pro and Canon 20 Da. With the latest generation of DSLR cameras, such as the Canon 1D Mark III and 40D, and the Nikon D3 and D300, live focus is becoming the standard.

The Live View video can usually be enlarged which greatly facilitates focusing on a subject. With respect to faint subjects, some methods focus on the faintest subject that is displayed on the Live View video display.

In some cameras it is possible to change the brightness of the display by changing the ISO. Other cameras automatically adjust the brightness to give the best display, although this is really intended for sunlit scenes. This live display can be accessed through the video out plug of the camera and fed to a monitor or computer. Any information that is displayed on the LCD on the back of the camera, such as menu items and picture review, can also be viewed through the video-out plug.

Live Video Out to a Television—The live video feed out of the camera can be fed to an auxiliary monitor or television where it can be viewed in a more comfortable position if the scope is pointed overhead where viewing the LCD on the back of the camera would be in an awkward position. A computer in the field is not necessary to use this feature. An old hand-held TV will prove satisfactory as a display. For example, a $15 black-and-white television that runs on D-cell batteries and 12 volts, DVD players, hand-held video game players, etc. can provide a display. Any device that has an input for analog video, for example, can be used to display the live video out of a DSLR.

Live Video Out to a Computer—Live video can be viewed in real time on a computer. Several devices are made for PCs for taking analog video feed and inputting it to a computer through a USB or Firewire connection, such as a Dazzle Digital Video Creator or Belkin Video Bus II.

Once hardware has been configured, the live video feed may be provided into a computer where a software program can be configured to view it. Several different freeware programs, such as VirtualDub, or Hocus Focus, are available.

Hocus Focus is a program designed by Gregory A. Pruden especially to focus a digital snapshot camera, such as a Nikon Coolpix that has real-time video-out capabilities, but it can also work with the Canon 20 Da. This program is very useful for focusing a subject, for example, because it has several different metric displays for focusing, such as Full Width Half Maximum (FWHM), maximum brightness, and radial sum value.

USB Out to a Computer—The latest generation of DSLR cameras (e.g., Canon EOS 40D and 1D Mark III, and Nikon D3 and D300) can feed the video out of the camera through the USB cable which also control the functions of the camera such as shutter speed, ISO, etc. These cameras also allow for remote viewing on a computer through camera control software that comes with the camera.

Live View focusing is the easiest method of focusing. It can be very accurate since it uses the actual image right off the sensor, viewed in real time. Its accuracy depends on user vision and judgment of correct focus.

Software Metrics

Computer programs may comprise focusing modules. A computer or laptop may be used to run the computer programs at the scope. With these programs, a subject, for example, is selected in the field and just that portion of the frame is downloaded. The computer then continuously downloads short exposures, and the subject is examined as the telescope, camera, imaging device, focusing system, or other optical instrument is focused. When the subject is at its smallest diameter or distances, the scope is in best focus. Some programs examine the subject and give a numerical readout of the subject diameter, FWHM, or maximum brightness value to assist in determining focus. Using this type of software, focus can be provided. It takes into account variables involved in focusing because it examines the actual final image on the chip.

A downside might be that the computer is set up next to the telescope, camera, imaging device, focusing system, or other optical instrument, which can be somewhat inconvenient if the scope is used in the field.

On some occasions, the viewed subject diameter can change during scintillation, making determination of the point of best focus problematic. In this case, a fainter subject can be used with longer exposures. Longer exposures average out deleterious effects, but take longer. Alternatively, software can take a number of exposures and average the values.

During the focusing process, the point of best focus may to be passed to confirm that it is, in fact, the point of best focus. Subsequently, focus is returned back to the point of best focus. It helps to pay attention to the numerical readouts in this case and note the numbers for best focus, and then try to find that position of the focuser again by trial and error. Note that these numbers can change, even if the focus is not moved.

A subject that is selected for use in focusing should not be saturated, especially if the brightness readout is used to determine best focus. The ISO or exposure time may be changed to ensure that the subject is not overexposed, depending on the brightness of the subject.

Software-assisted metric focusing is a very accurate method of focusing if viewing is reasonably good or, if viewing is not reasonably good, then averaging multiple exposures for each focus position is provided. It is a good method because it uses the actual subject image on the sensor; numbers that are an objective method of evaluating focus; and it does not rely on user eyesight.

Focusing Camera Lenses

Most modern auto-focus camera lenses will focus a little bit past infinity. This can present difficulties in focusing on subjects at “infinity,” especially in dark conditions. Some of these lenses are capable of focusing on a subject with the camera auto-focus mechanism, but the accuracy goes down as the focal length of the lens gets shorter, and the aperture of the lens gets smaller. In addition, many zoom lenses are not par-focal throughout the focal lengths of the zoom lens. This means that each focal length may focus at a different point for infinity focus.

On auto-focus lenses, the true point of infinity focus does not always coincide with the infinity mark on the focus distance indicator on the lens barrel. This is particularly true for zoom lenses, especially those that are not par-focal throughout the zoom range.

The longer the focal length of the lens, the more prone the lens can be to focus shift due to temperature changes.

Lenses in the Lens

A camera lens is actually several lenses combined into one unit. A single converging lens could form a real image on the film, but it would be warped by a number of aberrations.

One of the warping factors is that different colors of light bend differently when moving through a lens. This chromatic aberration essentially produces an image where the colors are not lined up correctly.

Cameras compensate for this using several lenses made of different materials. The lenses each handle colors differently. When combined in a certain way, the colors are realigned.

In a zoom lens, different lens elements can be moved back and forth. By changing the distance between particular lenses, the magnification power and/or the focal length of the lens as a whole can be adjusted.

Indirect Ophthalmoscopy and Retinal camera

Ophthalmic photography can include, for example, a system of information transferral. In fundus photography, a visual description of the three-dimensional retinal and choroidal tissues is transferred to film. This two dimensional representation is used by a physician to make judgments concerning the health and treatment of a patient's retina. An accurate representation of the retinal and choroidal tissues is the goal of the ophthalmic photographer. To this end, a fundus camera image should be sharp and well-focused. It is noted that the observation and photography systems of the fundus camera rely on the principle of indirect ophthalmoscopy

Indirect Ophthalmoscopy

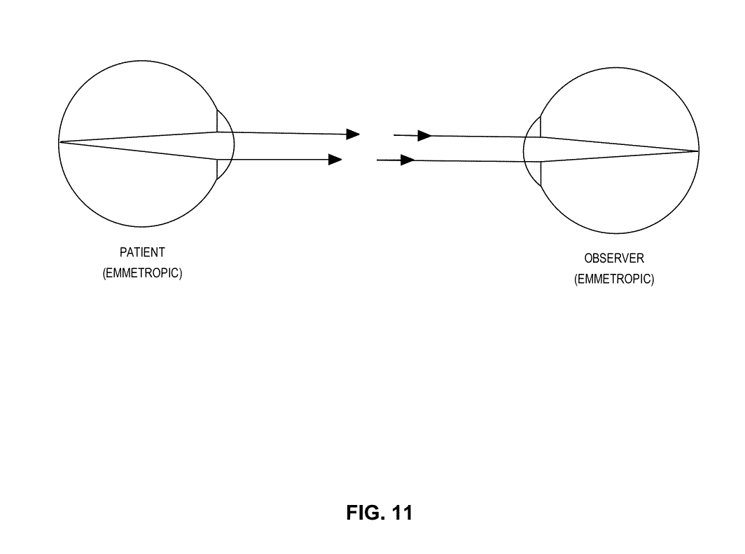

Imaging in direct ophthalmoscopy is illustrated in FIG. 11. If a patient's eye is emmetropic, light rays emanating from a point on the fundus emerge as a parallel beam. If this beam enters the pupil of an emmetropic observer, the rays are focused on the observer's retina and form an image of the patient's retina on the observer's retina. In short, if the patient and the observer are both emmetropic, rays emanating from a point in the patient's fundus will emerge as a parallel beam and will be focused on the observer's retina. This is called direct ophthalmoscopy.

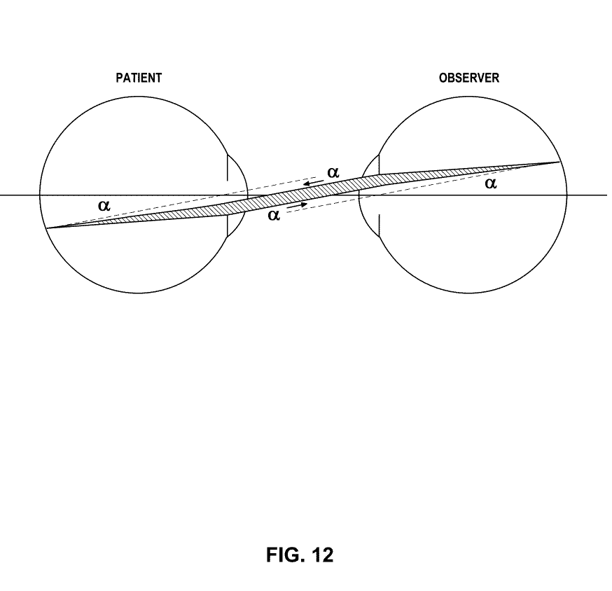

If the patient's fundus is properly illuminated, the field of view is limited by the most oblique pencil of light that can still pass from the patient's pupil to the observer's pupil as illustrated in FIG. 12. In direct ophthalmoscopy, the retinal point that corresponds to this beam can be found by constructing an auxiliary ray through the nodal point of the eye. The point farthest from the centerline of view that can still be seen is determined by the angle α, that is, the angle between this oblique pencil and the common optical axis of the eyes. The maximum field of view is determined by the most oblique pencil of rays (shaded) that can still pass from one pupil to the other.

Angle α, and therefore the field of view, is increased when the patient's or the observer's pupil is dilated or when the eyes are brought more closely together. Even with appropriate illumination, direct ophthalmoscopy has a small field of view.

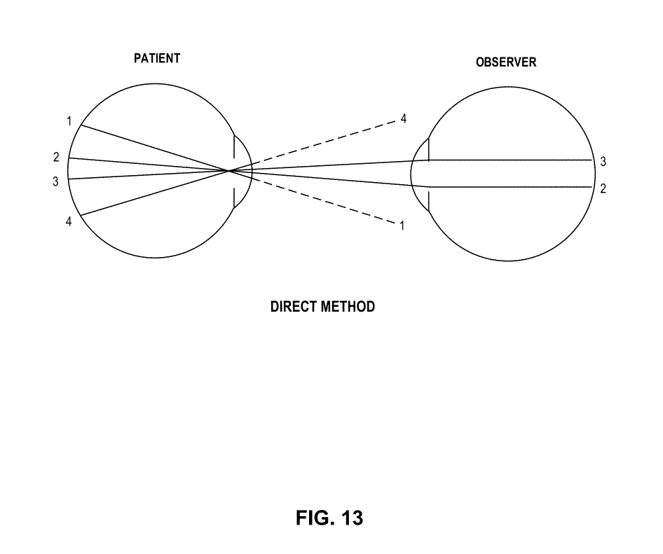

FIG. 13 shows a limited field of view in the direct method. Peripheral pencils of light do not reach the observer's pupil. Of the four points in the fundus, points one and four cannot be seen because pencils of light emanating from these points diverge beyond the observer's pupil. To bring these pencils to the observer's pupil, their direction can be changed.

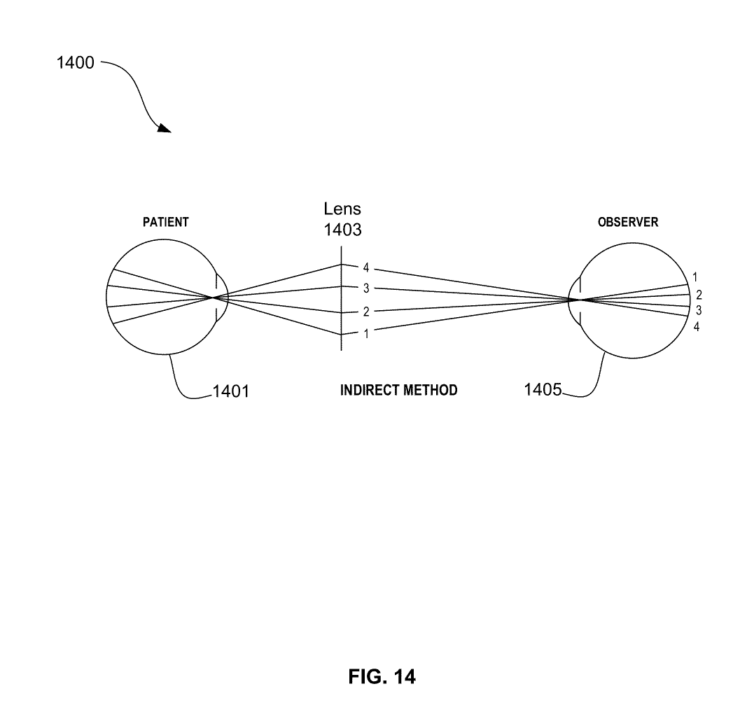

FIG. 14 shows an extended field of view in the indirect method. The ophthalmoscopy lens redirects peripheral pencils of light toward the observer. A fairly large lens is located somewhere between the patient's and the observer's eye. This principle was introduced by Ruete in 1852 and is called indirect ophthalmoscopy to differentiate it from the first method, in which the light traveled in a straight, direct path from the patient's eye to the observer. The use of the intermediate lens 1401 has several implications that make indirect ophthalmoscopy more complicated than direct ophthalmoscopy.

A purpose of the ophthalmoscopy lens is to bend pencils of light toward the observer's pupil as illustrated in FIG. 14. FIG. 14 also demonstrates a characteristic side effect of this arrangement: compared with the image in direct ophthalmoscopy, the orientation of the image on the observer's retina is inverted. For the novice, this often causes confusion in localization and orientation. FIG. 14 shows that in this arrangement the patient's pupil is imaged in the pupillary plane of the observer. In optical terms the pupils are in conjugate planes.

The field of view in indirect ophthalmoscopy is determined by the rays emerging from the patient's eye that can be caught in the ophthalmoscopy lens. With optimal placement of the lens and of the observer's eye 1405, the distance from the patient's eye 1401 to the lens 1403 is only slightly more than the focal length of the lens 1403. The exact distance can be calculated as will be shown below. The field of view is thus determined by the ratio of lens diameter and focal length. This ratio can also be written as a product:

(Lens diameter)/(Focal length)=(Lens diameter)×(dioptric power).

This provides the relationship for comparing the field of view of various lenses.

Given lenses of equal power, a larger lens provides a wider field of view. If lenses have equal diameters, a stronger lens provides a wider field of view. However, because stronger lenses often have a smaller diameter, a stronger ophthalmoscopy lens does not always provide a larger field. For example, a 20-diopter (D) lens of 30 mm provides about the same field of view as a 30-D lens of 20 mm or as a 13-D lens of 45 mm (because 20×30=30×20=±13×45).

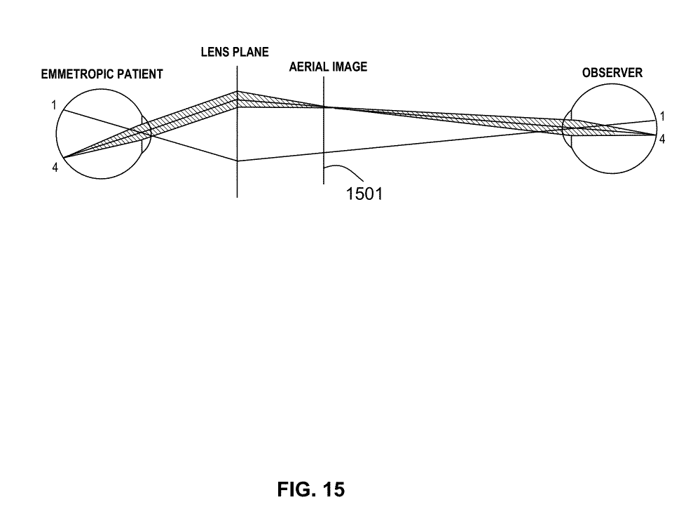

FIG. 14 shows that light emerging from the patient's fundus is directed toward the observer's eyes. It does not specify whether the observer sees a focused image or just an unstructured red reflex. FIG. 15 traces the rays within one of the pencils of light from the patient's fundus to the observer's retina. FIG. 15 shows imaging in the indirect method. A pencil of rays (shaded) is traced from the patient's fundus to the observer's retina. An intermediate real image 1501, which is an inverted image, of the patient's fundus is formed in the focal plane of the ophthalmoscopy lens. The observer can accommodate on this image.

If the patient is emmetropic, the pencils emerging from the eye are composed of parallel rays, but this changes once the pencils pass through the ophthalmoscopy lens. In fact, because the rays within each pencil enter the ophthalmoscopy lens with zero vergence, they are brought to a focus in the focal plane of the ophthalmoscopy lens. Proceeding beyond that point, the rays within each pencil are divergent.

Considering all pencils emerging from the patient's eye together, an aerial image of the patient's fundus will be formed in the focal plane of the ophthalmoscopy lens. This image is inverted with respect to the patient's fundus, and it is this image that the observer is viewing. To focus the aerial image on his or her own retina, the observer must accommodate for the aerial image plane and hence cannot approach too closely.

It may be useful to note the difference between tracing of pencils and tracing of rays. In any optical system, the tracing of pencils is used to determine the limits of the field of view, while the tracing of rays is necessary to determine the position of the image plane. Optical diagrams may confuse the uninitiated, because they generally trace only one ray per pencil (see, e.g., FIGS. 13 and 14) and may use theoretic auxiliary rays beyond the physically existing pencils (see, e.g., FIG. 12) to facilitate the construction of object and image planes.

In direct ophthalmoscopy, peripheral pencils of light are increasingly cut off by the observer's and patient's pupils (see, e.g., FIG. 12). In indirect ophthalmoscopy (see, e.g., FIG. 15), this does not happen; only the observer's pupil limits the diameter of the pencils that reach the observer's retina. The apparent luminosity of the fundus image, therefore, is constant throughout the field, provided, of course, that the fundus is illuminated evenly. This is one of the reasons why fundus cameras are built around the imaging principle of indirect ophthalmoscopy.

In summary, the purpose of the ophthalmoscopy lens in indirect ophthalmoscopy is to redirect diverging pencils of light emerging from the patient's pupil toward the observer's eye. In doing so, the lens also focuses parallel rays within each pencil into an inverted aerial image of the patient's fundus. The existence of an inverted aerial image is a prominent and unavoidable characteristic of indirect ophthalmoscopy.

The indirect method offers a wider field of view than direct ophthalmoscopy. However, this advantage can be at the expense of decreased magnification.

Magnification in indirect ophthalmoscopy can best be understood if broken down into two components: magnification from fundus detail to aerial image and magnification from aerial image to the observer's retinal image. Magnification in the first step depends on the power of the ophthalmoscopy lens. Magnification in the second step depends on the observation distance.

If the patient is emmetropic, the aerial image is formed in the focal plane of the lens (compare FIG. 15).

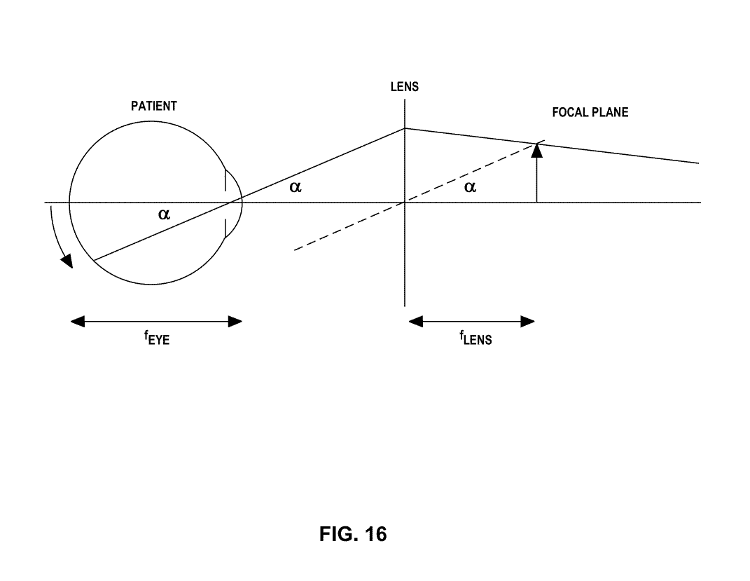

FIG. 16 shows a magnification of the aerial image in the indirect method. Aerial image size is found through construction of an imaginary, auxiliary ray (dotted line). Fundus detail is f_{eye}×sin α. Aerial image is fₗₑₙₛ×sin α.

FIG. 16 shows that

(Aerial image)/(Fundus detail)=(fₗₑₙₛ×sin α)/(f_{eye}×sin α)=fₗₑₙₛ/f_{eye},

converting from focal length to diopters and assuming 60 D as the power of the eye:

(Aerial image)/(Fundus detail)=(fₗₑₙₛ×sin α)/(f_{eye}×sin α)=fₗₑₙₛ/f_{eye}=D_{eye}/Dₗₑₙₛ=60/(lens power).

Thus, the aerial image formed by a 20-D lens is 60/20, or three times larger than the corresponding fundus detail; with a 30-D lens it is 60/30, or two times larger.

When the aerial image is viewed from 25 cm, no further magnification is involved, because 25 cm is the reference distance for magnification. A 25-cm viewing distance from the aerial image uses 4 D of accommodation on the part of the observer; a more common viewing distance is 40 cm, using 2.5 D of accommodation. Changing from 25 cm to 40 cm reduces the observer's retinal image size by 25/40 or ⅝.

Combining both steps, the following is obtained. With a 20-D lens and a distance of 25 cm from aerial image to observer, the patient's disc is seen as 3 times larger than the disc of a dissected eye at 25 cm. With direct ophthalmoscopy, this would have been 15 times larger. In this case, indirect ophthalmoscopy provides five times less magnification than does direct ophthalmoscopy. For a 40-cm viewing distance the magnification is ⅝×3, which is approximately 2, or 8 times less than direct ophthalmoscopy.

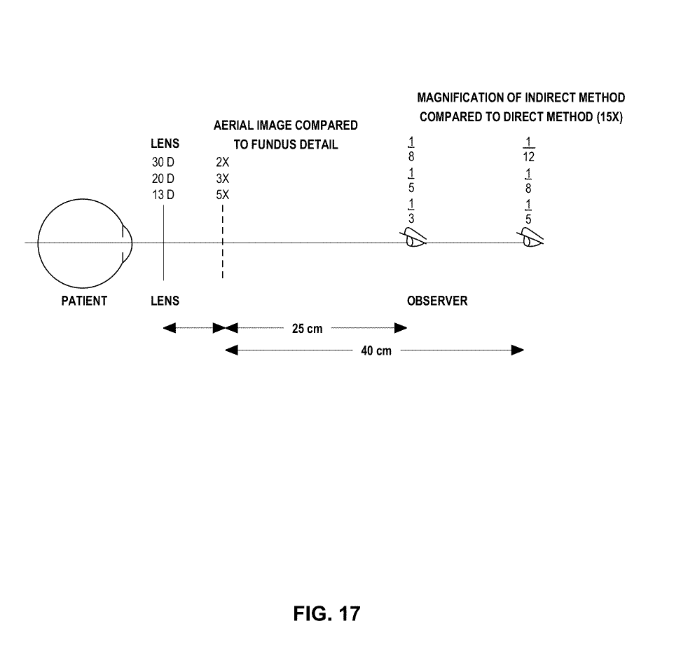

Similar calculations can be made for other lenses. FIG. 17 summarizes data for lenses of 30 D, 20 D, and 13 D. As the magnification becomes less, the area of the patient's fundus that can be imaged on a given area of the observer's retina increases quadratically. For example, 8 times less linear magnification potentially results in a 64-times larger area being seen. Whether this potential is realized depends on the factors mentioned in relation to the field of view in both methods: width of the illuminating beam in direct ophthalmoscopy and diameter of the ophthalmoscopy lens in indirect ophthalmoscopy.

FIG. 17 shows a total magnification in the indirect method. Total magnification depends on lens power and observation distance and is less than (e.g., always less than) in the direct method.

In summary, in indirect ophthalmoscopy, the observer's retinal image is considerably less magnified than in direct ophthalmoscopy. The stronger the lens, the less magnified is the image. This is the price paid for the enlargement of the field of view. For any given ophthalmoscopy lens, some extra magnification can be gained by reducing the viewing distance, but this employs extra accommodation by the observer.

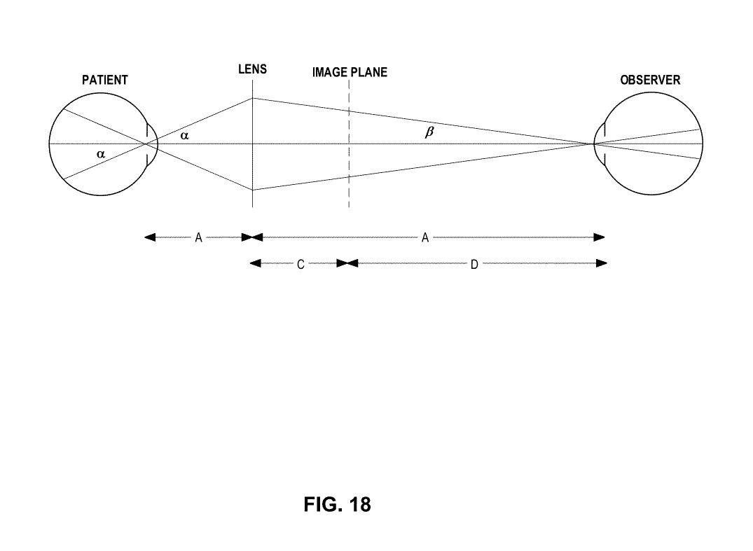

An alternative calculation compares the size of the observer's retinal image with the size of the patient's fundus detail. This ratio can be 1:1 in direct ophthalmoscopy. This calculation, which bypasses the size of the aerial image, is explained in FIG. 18 which has an accompanying table. The observer's retinal image size and the size of the corresponding fundus detail in the patient are determined by the angles α and β. These, in turn, are proportional to the distances a and b. The relationship of a and b follows from a constraint that the patient's and the observer's pupils must be in conjugate planes (in diopters: 1/a+1/b=lens power).

The table below shows calculations of distances and magnification for various lenses and viewing distances in the indirect method.

| Patient | Lens | c (cm) | d (cm) | b (cm) | 1/b | 1/a | a (cm) | a/b | a + b (cm) |
|---|---|---|---|---|---|---|---|---|---|
| Emm | 13D | 7.7 cm | 25 | 32.7 | 3.1D | 9.9D | 10.1 | 1/3 | 43 |
| Emm | 13D | 7.7 cm | 40 | 47.7 | 2.1D | 10.9D | 9.2 | 1/5 | 58 |
| Emm | 20D | 5 cm | 25 | 30 | 3.3D | 16.7D | 6.0 | 1/5 | 36 |
| Emm | 20D | 5 cm | 40 | 45 | 2.2D | 17.8D | 5.6 | 1/8 | 51 |
| Emm | 30D | 3.3 cm | 25 | 28.5 | 3.5D | 26.5D | 3.8 | 1/8 | 32 |
| Emm | 30D | 3.3 cm | 40 | 40 | 2.3D | 27.7D | 3.6 | 1/12 | 47 |

The steps in the calculation are as follows. Given the patient's refractive error and the lens power, the distance c from the lens to the aerial image can be calculated. If the patient is emmetropic, c is the focal length. Given the observer-to-aerial-image distance (d), b can be calculated (b=c+d). Given b, a can be calculated, and subsequently a/b and a+b.

The last column (a+b) indicates the total distance from patient to observer. The a/b ratio indicates the ratio of the observer's retinal image to the patient's fundus detail. In direct ophthalmoscopy, this ratio can be 1:1. The values in this column thus are the same as those in FIG. 17. The a/b ratio can be used in calculations relating to the latitude of beam placement.

Through these calculations the field of view formula can be refined. The proper formula is lens diameter/a instead of lens diameter/focal length. The actual value of a, and hence the field of view, varies somewhat with the patient's refractive error and the observer's viewing distance. The effect of this refinement is small and the earlier formula remains a useful rule of thumb.

Compensation for Refractive Error

Some discussions above contemplate that both the subject and the observer are emmetropic. This is not always the case.

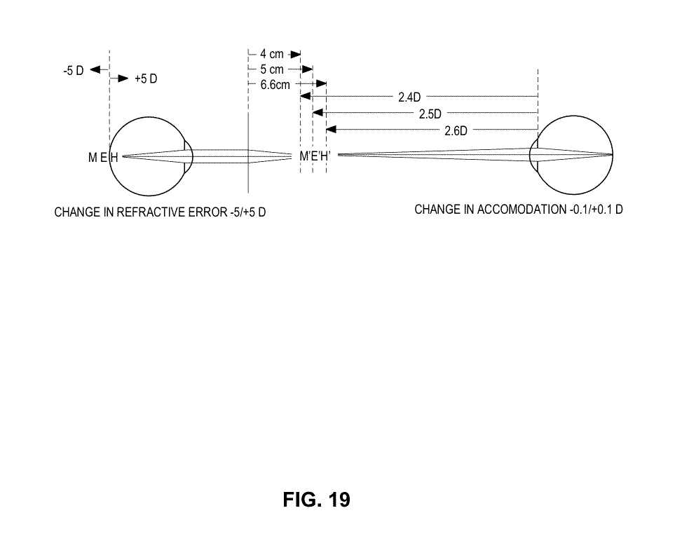

In direct ophthalmoscopy, the problem can be overcome by having the patient and the physician wear their respective spectacle (or contact lens). Each eye with its correction then acts as an emmetropic system. This method can be used, for example, in cases of high refractive errors and in cases of marked astigmatism. For small refractive errors, however, it is useful to remove the glasses so that the eyes can then be approached more closely, resulting in an increased field of view. A single lens in the ophthalmoscope can replace the mathematical sum of the patient's and the observer's correction. In one example, Rekoss (1852) devised a system of two disks (e.g., one carrying lenses with large steps and one with small steps), a miniature version of the disks used in a phoropter. In indirect ophthalmoscopy, compensation for refractive error can be made without additional lenses. The data for FIG. 19 is from a recalculation of the table above for a 20-D lens, 45 cm between the lens and the observer, and various degrees of patient ametropia.

FIG. 19 illustrates compensation for refractive error. With the indirect method, minor changes in the observer's accommodation can compensate for major changes in the patient's refractive error.

If the patient is emmetropic (E), the aerial image (E′) will be 5 cm from the lens. If the observer is 45 cm from the ophthalmoscopy lens, the observer accommodates for 40 cm (2.5 D). If the observed fundus detail lies in a plane (M) representing 5 D of myopia, the aerial image (M′) will be at approximately 20+5=25 D=4 cm. The accommodation used is for 41 cm (2.45 D). A fundus detail representing 5 diopters of hyperopia (H) forms an aerial image (H′) at 20−5=15 D=6.6 cm, so that an accommodative increase to 38.3 cm (2.6 D) is used. Thus, minor changes in the observer's accommodation can account for major refractive errors that the patient may have. The presbyopic observer, who cannot change accommodation, can compensate for the patient's refractive error by changing the observation distance or by using a near-vision addition.

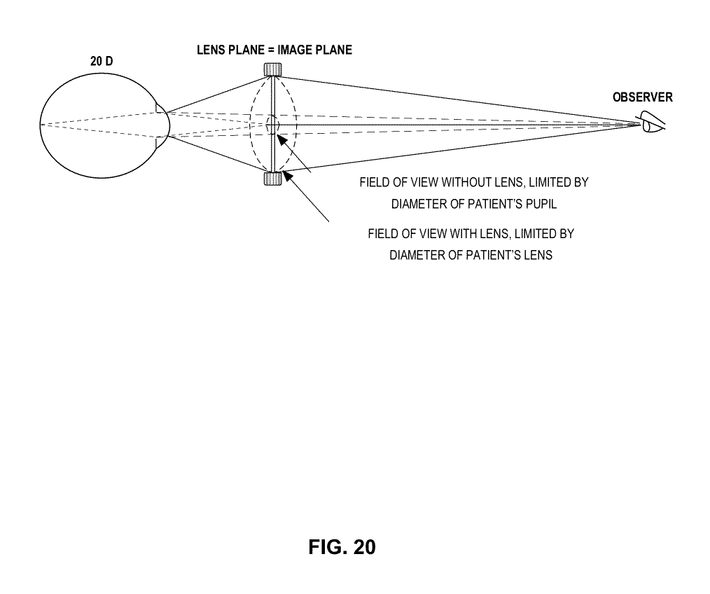

An interesting example occurs for a patient with 20-D myopia. In this instance, the eye forms its own aerial image at 5 cm, that is, in the plane of the ophthalmoscopy lens. The ophthalmoscopy lens does not change the location of this image. This image can be viewed without the ophthalmoscopy lens, but the field of view in is limited to the patient's pupil as illustrated in FIG. 20. FIG. 20 shows indirect ophthalmoscopy of a high myope. The myopic eye forms its own aerial image (dotted lines) without the help of the ophthalmoscopy lens. Without the lens, only the central part of this image is visible (dashed lines, limited by the patient's pupil). With the lens (solid lines), the image is limited by the lens rim. Also, with the lens, the field of view becomes far larger. Thus, the field-enlarging function of the ophthalmoscopy lens can be separated from its aerial image-forming function. Other examples can be found using contact lens methods.

Focusing System of the Fundus Camera and Fundus Imaging System

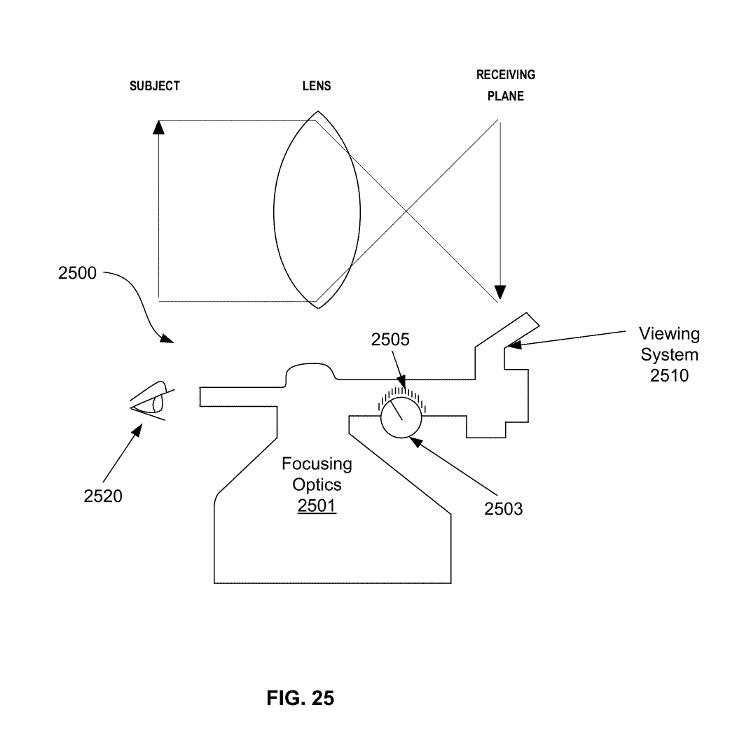

Fundamentally, optical systems include light, a subject, a lens, and a receiving plane. Light reflects off of the subject, is refracted by the lens, and is projected onto a receiving plane as an image. Clinically focusing the fundus camera includes adjusting the relationship between the subject and lens so that the subject lies within the lens' depth of field and the receiving plane lies within the depth of focus of the image. In fundus photography, the patient's retina becomes the subject, the optics of the fundus camera and the patient's eye replace the simple lens, and the film or other recording media becomes the receiving plane as shown in FIG. 25.

Most fundus cameras use a single lens reflex viewing system. An operator's (e.g., a physician's) focusing skills are enhanced with familiarity of the single lens reflex (SLR) design and the fundus camera's aerial image viewing system.

The SLR Viewing System

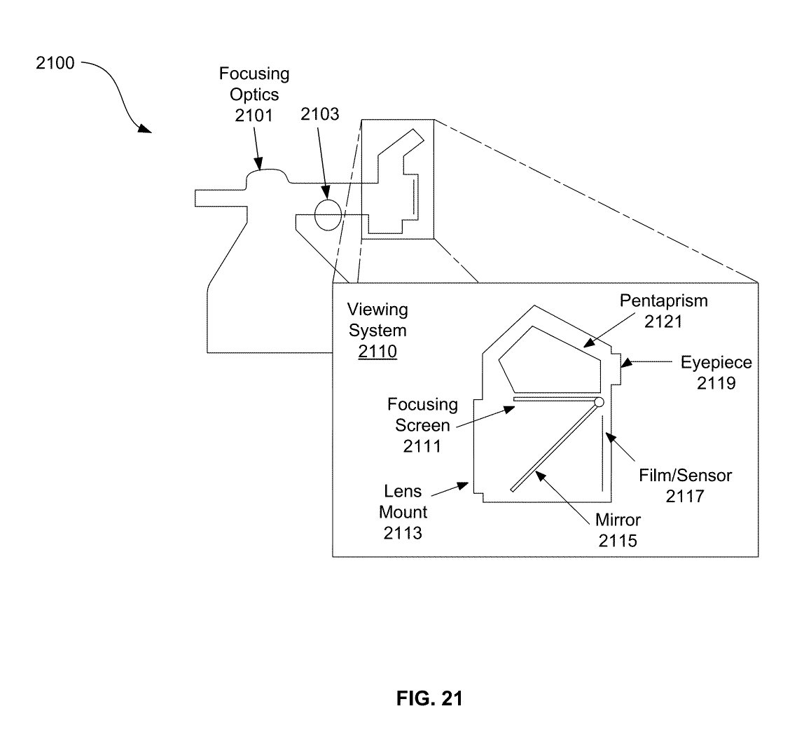

FIG. 21 shows an example of the fundus camera's SLR viewing system. Some, but not all, components (e.g., standard components) of the SLR camera system are identified. Referring to FIG. 21, there is shown a fundus camera 2100 comprising focusing optics 2101, a focusing mechanism 2103, and a viewing system 2110. Most modern fundus cameras use a single lens reflex (SLR) system for viewing the fundus image. The objective lens of the fundus camera transmits light for both viewing the subject and exposing the film or sensor inside the SLR camera body. In an example scenario, the fundus camera system 2100 comprises an indirect ophthalmoscopic device.

The focusing mechanism 2103 may comprise a focusing knob, for example, but is not limited to a physical knob structure, and may instead comprise an electronic control that configures lens movement through electric motors or piezoelectric actuators, for example. The focusing optics 2101 may comprise one or more movable lenses that may be moved laterally to modify the focal point of the fundus camera 2100.

The viewing system 2110 may comprise a focusing screen 2111, a lens mount 2113, a hinged mirror 2115, film/sensor 2117, an eyepiece 2119, and a prentaprism 2121.

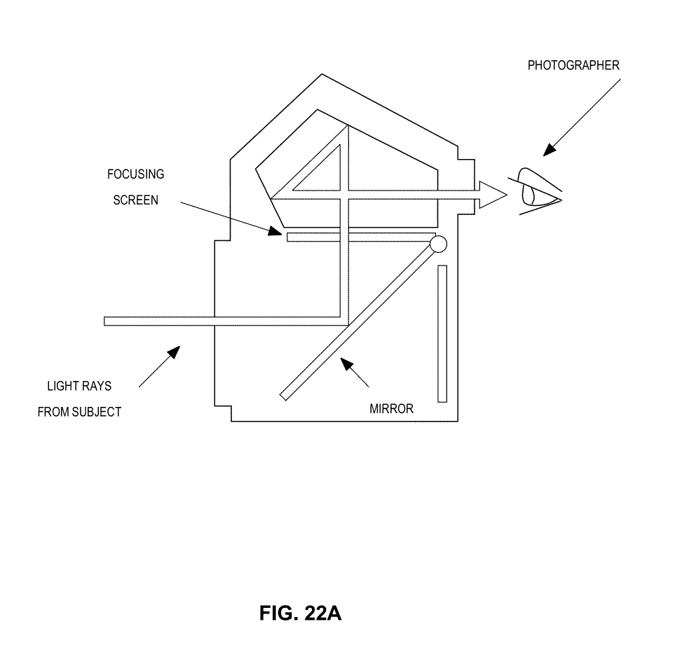

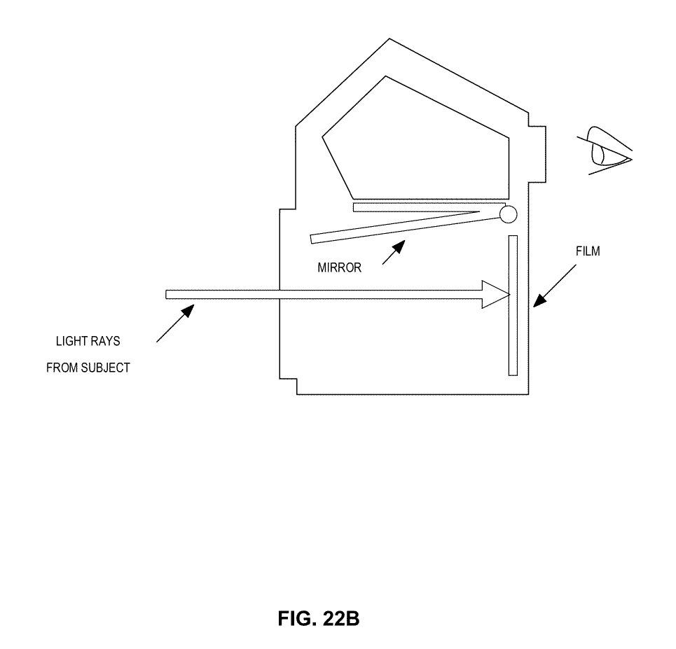

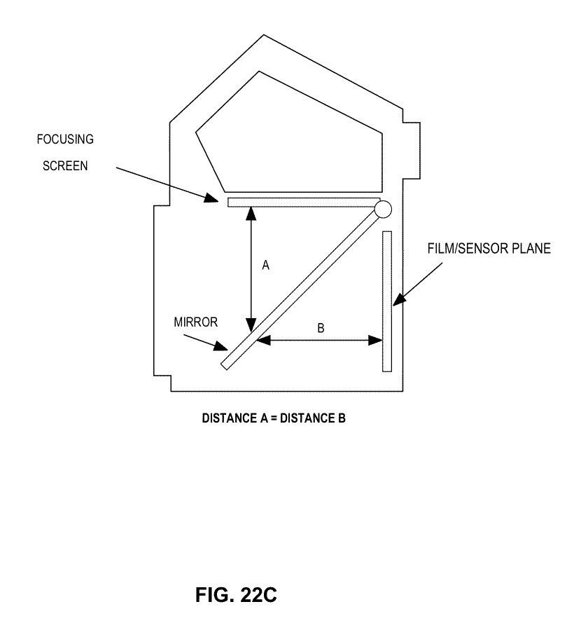

The hinged mirror 2115 is positioned differently for viewing and taking the picture as shown in FIGS. 22A-B. The image is seen on the focusing screen 2111 via the eyepiece 2119 when the mirror 2115 is down. The hinged mirror 2115 then flips up and out of the way to allow film or sensor 2117 exposure. In the SLR system, the focusing screen 2111 helps the photographer judge image sharpness before exposing the film or sensor for taking an image. The focusing screen 2111 may be constructed of ground glass and is located at the same distance from the objective lens as the film or sensor plane as shown in FIG. 22C. When an object appears sharp on the focusing screen 2111, it will also be sharply imaged at the film/sensor plane and therefore will be in sharp focus on the film or sensor 2117. The photographer focuses by adjusting the objective lens utilizing the focusing knob 2103 to achieve the sharpest image on the textured focusing screen 2111. Unfortunately, even when an SLR system incorporates the finest ground glass manufactured, the intensity of the light decreases as it passes through the focusing screen 2111. A ground glass focusing system dims the view of the object being photographed. This darkening of the focusing screen 2111 is of little importance to most photographers. However, in fundus photography, a number of restrictions can preclude the use of a ground glass focusing system including, for example, one or more of the following: relatively small amounts of light used when viewing the eye; high magnifications used to photograph the retina; and the grainy structure of a ground glass focusing screen breaks up the fine detail of a fundus image.

Before a photograph is taken, the photographer previews the scene. Light from the lens enters the camera body, and reflects off of the hinged, 45° mirror and up into the focusing screen. The photographer views the image on the focusing screen through the viewfinder as shown in FIG. 22A. When the photographer chooses to expose an image, the shutter release is depressed. This initiates a sequence of events (these events may vary according to the specific camera and flash utilized) which includes the mirror flipping up and out of the way, and a momentary darkening of the viewfinder. This allows the same light which has just been viewed to expose the film or sensor as shown in FIG. 22B. The SLR system may be considered a “what-you-see-is-what-you-get” system. The distance between the focusing screen and the lens system is equal to the distance between the film/sensor plane and the lens system as shown in FIG. 22C. When the image appears sharp on an SLR camera's focusing screen, it will also appear sharp on the film or sensor output.

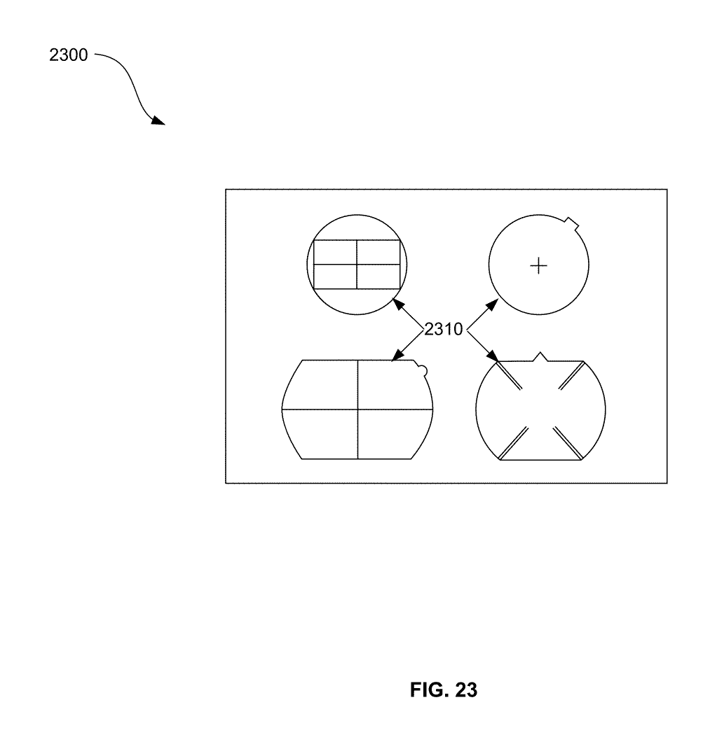

Some fundus cameras replace the ground glass of a standard 35 mm SLR camera system with a clear glass focusing screen with etched lines as illustrated in FIG. 23. The clear glass may maximize the light entering the photographer's eye. The etched black lines 2310 (called the fusing reticle) allow the photographer to focus on the plane of the clear glass focusing screen 2300 and to achieve sharp focus at the film/sensor plane. FIG. 23 shows various designs of focusing plane reticles. The reticle for a specific camera can vary according to manufacturer and date.

Methods and Examples

Determination of Ocular Refraction

The dioptric power (K) of the ametropia, or refractive error, of the eye is equal to 1/k (i.e., K=1/k) in diopters, according to paraxial theory, where k is the distance in meters between the far point and either the spectacle plane (spectacle refraction), or the principal point of the eye, or the refracting surface of the reduced eye (ocular refraction).

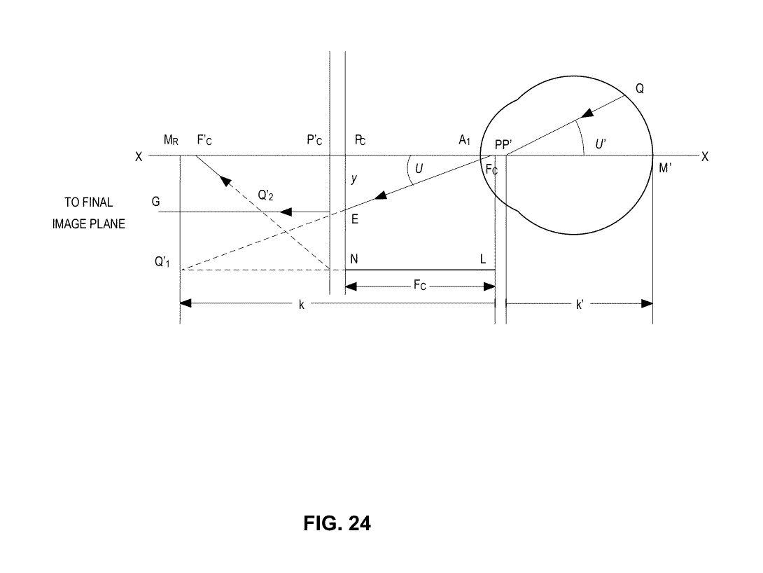

According to the model illustrated in FIG. 24, k is the distance from P to the eye's far point (M_{R}) and k′ is the distance from P′ to the retina (M′). Ocular refraction (K) is the reciprocal of the distance k. This convention has been adopted for all future references to k and k′.

FIG. 24 illustrates, inter alia, the telecentric principle for a myopic three-surface eye. The anterior principal focus F_{C} of the condensing lens is intended to coincide with P, the eye's first principal point; k is the distance from P to the far point plane (M_{R}); f_{c} is the anterior focal length of the camera imaging system; k′ is the distance from the eye's second principal point to the retina; t is the height of a retinal feature; and A₁ is the corneal vertex. Hence, the true nature of the factor q does not emerge. Bengtsson and Krakau describe the process of aligning the Zeiss Oberkochen fundus camera in front of a human eye to ensure that it is in the correct position for the telecentric principle to apply.

Some of the features of the telecentric design are illustrated in FIG. 24, in which a subject's eye has its first and second principal points at P and P′. The components of the camera imaging system are represented as a thick lens, with anterior and posterior principal points at P_{C} and P′_{C}, respectively.

Telecentric Principle and Ocular Refraction

As noted above, ocular refraction (K) is the reciprocal of the distance k (i.e., K=1/k).

Refractive Power

Refractive power (F) can be described as the ability of a lens or an optical system to change the direction of a pencil of rays. It is equal to

F=n′/f′=−n/f

where n and n′ are the refractive indices of the object and image space, respectively; f and f′, the first and second focal length, respectively, in meters; and the power F is expressed in diopters. Refractive power can be synonymous with dioptric power, focal power, and vergence power.

Paraxial Equation

The paraxial equation is based on Gaussian theory and deals with refraction at a spherical surface:

$$\frac{{n}^{′}}{{l}^{′}} - \frac{n}{l} = \frac{{n}^{′} - n}{r} ( or ) {L}^{′} L = \frac{{n}^{′} - n}{r}$$

where n and n′ are the refractive indices of the media on each side of the spherical surface; r is the radius of curvature of the surface; l and l′, the distances of the object and the image from the surface, respectively; n/l and n′/l′, the vergences (or reduced vergences) of the incident and refracted light rays, respectively; and L′−L corresponding to the change produced by the surface in the vergence of the light and being called the focal power F (or vergence power, refractive power, etc.) of the surface. The vergence of a light ray is the reciprocal of the distance between the point of focus and a reference plane. Thus, L′−L=F.

Focal power can be expressed in diopters and can be positive or negative. At a reflecting surface or a mirror, the equation becomes

L′−L=2/r

where r is the radius of curvature of the surface or mirror.

Surface Power

Surface power is the dioptric power of a single refracting or reflecting surface. It is equal to

F=(n′−n)/r

where F is the power in diopters, n and n′ are the refractive indices of the media on each side of the surface and r is the radius of curvature of the lens or mirror surface, in meters. This equation forms part of the fundamental paraxial equation. For a spectacle lens in air (n=1) the power of the surface becomes

F=(n′−1)/r

As an example, the following is a calculation for a power of an anterior corneal surface:

$$F = \frac{1.376 - 1}{0.0078} = 48.21 D$$

where the refractive index of the cornea is 1.376 and the surface has a radius of curvature of 7.8 mm. In another example, the following is a calculation for a power of a posterior corneal surface:

$$F = \frac{1.336 - 1.376}{0.0067} = - 5.97 D$$

where the refractive indices of the aqueous humor and the cornea are 1.336 and 1.376, respectively, and the surface has a radius of curvature of 6.7 mm. For a thin spectacle lens in air, the sum of the powers of the two surfaces F1+F2 represents the total power of the lens and is equal to

$$F = {F}_{1} + {F}_{2} = ( n - 1 ) ( \frac{1}{{r}_{1}} - \frac{1}{{r}_{2}} )$$

Additional Metrics

The ocular refraction K can be calculated using a far point distance k. If k is in meters, then K is in diopters (D):

K=1/k and k=1/K.

Axial length and dioptric length are given by

k′=nₑ/K′ and K′=nₑ/k′.

Ocular refraction for a reduced surface power and dioptric length is given by

K′=K+Fₑ.

Focal length and power of reduced surface:

f′ₑ=nₑ/Fₑ and Fₑ=nₑ/f′ₑ,

where f′ₑ is in meters.

Equations for vertex distance compensation are set forth below. For example, if the vertex distance is decreased, then

F_{new}=F_{old}/[1−(dF_{old})].

If the vertex distance is increased, then

F_{new}=F_{old}/[1+(dF_{old})].

Focusing Technique Using Fundus Imaging System

Some embodiments of focusing techniques or focusing systems employ retinal fundus imaging systems to acquire, for example, information or data to determine the refractive errors of a subject (e.g., a patient).

Littman developed a technique for determining the true size of a given fundus feature for the Zeiss Oberkochen telecentric fundus camera. Assuming axial ametropia, some embodiments of the focusing techniques use Littmann's formula to derive a refractive status of an eye.

Littmann's formula is

t=1.37qs (1)

The formula relates the true size t of a retinal feature to the measured size s of its image on the fundus camera film. In this example, an accurate ocular refractive state of the examined eye is used in which q is a factor dependent on optical dimensions of the eye. Thus, a clear focal image is used to acquire a true size of a retinal feature. The coefficient 1.37 is a constant specific to the Zeiss Oberkochen instrument used by Littmann. A different fundus camera can have a different coefficient p instead of 1.37. Littmann did not explain the rationale of his procedure and its dependence on a telecentric camera system.

Some embodiments of focusing techniques or focusing systems that employ retinal fundus imaging systems are described below.

Referring to FIG. 24, the anterior principal focus F_{c} of the camera imaging system is configured to be coincident with P. Any emergent ray from the fundus, such as PE, becomes parallel to the optical axis after refraction by the camera's imaging system. The size of the image on the camera film plane is directly proportional to y, the distance P_{c}E, which is governed by the angle U, which in turn is proportional to U′. A ray QP′ from a retinal point Q gives rise to the refracted ray PE, which meets the camera's imaging system at E. On refraction by the eye itself, an image Q′₁ forms in the plane of the eye's far point M_{R}, which is conjugate with the retina. Q′₁ lies on the path of the refracted ray PE produced backward to the far point plane. Next, Q′₁ becomes an object for the camera's imaging system. After refraction by this lens, the emergent ray EG will be parallel to the optical axis.

At this stage, the second image of Q lies along EG. Its location can be determined as set forth below. A construction line NL parallel to the optical axis XX directed toward Q′₁ will be refracted through the posterior focal point of the camera imaging system (F′_{c}) and meet the refracted ray EG at the second image point Q′₂. Ray EG continues to the camera film plane or sensor.

According to paraxial theory, k is the distance from P to the eye's far point and k′ is the distance from P′ to the retina. Ocular refraction is the reciprocal of the distance k. The conjugate angles U and U′ in FIG. 24, if small, can be taken as obeying the simplified law of refraction, so that U′/n=U/n′, where n is the refractive index of the final ocular medium. This may be taken as 1.336, a generally accepted value.

The telecentric optical system may be designed such that the final image is at infinity, and the ratio of the image size on the camera film plane(s) to the distance y (P_{c}E) is a constant over a wide range of ametropia. Because the distance P_{c}P (which is equal to the anterior principal focal length of the camera imaging system) can be assumed to remain constant, the only variable governing y is the angle U between the refracted ray PE and the optical axis. The telecentric design thus gives rise to the relationship

t=p(0.01306k′)s (2)

Comparison of this expression with Littmann's equation—Equation (1) above—shows that 1.37 is the value of p for the Zeiss fundus camera used by Littmann, whereas the middle term is equivalent to the variable q such that

q=0.01306k′ (3)

which shows q to be a constant fraction (approximately 1/80) of the crucial ocular dimension k′. It is generally appreciated that k′ and q cannot be determined directly, but can only be evaluated to varying degrees of approximation.

In some configurations, the optical system of the actual-size model eye used has a cornea and crystalline lens made from a gas-permeable contact lens material (Boston RXD; Polymer Technology Corp., MA), which has a refractive index of 1.435. The equivalent powers of the cornea and crystalline lens are +43.90 D and +20.81 D, respectively. A hemispherical surface represents the fundus (radius=11.50 mm). Distilled water was used to fill the anterior and posterior chambers and, in this situation, the equivalent power of the model eye is +59.63 D. Full specifications of this model eye have previously been published in A. R. Rudnicka et al., “Construction of a model eye and its applications,” Ophthalmic Physiol Opt., 12(4):485-90 (October 1992). A square test object was applied to the fundus surface of the model eye. The fundus surface can be removed from the model eye, and this allowed the side length of the square (t) to be measured directly with a traveling microscope. The distance t was measured as a linear distance.

This is acceptable for relatively small dimensions, as in this case, but for larger dimensions, the curvature of the retina is taken into consideration. From Equations (2) and (3), the camera correction factor p is given by

p=t/(qs) (4)

Images were taken with the posterior chamber depth of the model eye set at values corresponding to a range of ocular refractions from +11 D to −14 D. In addition, the model eye was rendered aphakic by removing its crystalline lens and, by alteration of the posterior chamber depth, ocular refraction values from emmetropia to 120 D are possible. Between 66 D, images were recorded every 1 D and thereafter every 2 D. In all cases, the square object was centered in the camera field. The variable quantity q was calculated for the range of ametropia settings of the model eye, knowing its optical construction. Using Equation (4), the value for p was determined for each instrument at each setting. In the case of a telecentric system correctly aligned, p is constant irrespective of the ametropia. Kodak Ektachrome 200 film (Eastman-Kodak, Rochester, N.Y.) was used in the fundus cameras. For the fundus cameras, the image of the square target on Kodak Ektachrome slide film was measured under fixed magnification of 317.5 using a text reader fitted with a graticule. The side length of the image of the square target was measured to give the value for s.

The built-in correction factors used by laser imaging systems to correct image size measurements according to a patient's refractive error are applicable to a human eye, but might not be directly applicable to the model eye. Therefore, the following procedures were adopted. For the scanning laser ophthalmoscope and Heidelberg Laser Tomographic Scanner, images were stored as Tagged Image Format Files. The side length of the image of the square target was measured in pixels directly on a computer using a commercially available program for measuring Tagged Image Format File images, which gives values for s in pixels. Image size measurements with the Heidelberg Retina Tomograph were performed using a special version of the software that did not compensate for refractive error. This program gives values for s in millimeters.

k′=t/0.01306ps (5)

From earlier equations discussions,

K′=K+Fₑ

Thus,

Fₑ=K′−K.

Therefore,

K=−Fₑ/K′ (6)

Experimental Procedure

To record ocular refractions in accordance with some embodiments of the present disclosure, various determinations (e.g., measurements, calculations, etc.) are performed as shown in FIGS. 25-28.

FIG. 25 shows a fundus camera and fundus imaging system in a focusing system. Referring to FIG. 25, there is shown a fundus camera 2500 comprising focusing optics 2501, a focusing mechanism 2503 with a Vernier scale 2505, and a viewing system 2510. The fundus camera 2500 may be similar to the fundus camera 2100 described with respect to FIG. 21. The focusing mechanism 2503 may comprise a focusing knob or other actuating control mechanism for configuring the focusing optics 2501.

The position of the focusing mechanism 2503 on the fundus camera 2500 may be recorded. The position of the focusing mechanism 2503 reflects the optical error or refraction of the eye 2520. The refraction of the eye 2520 may be determined by calculating the object to image size ratio or magnification. This focus mechanism 2503 position is preferably automatically incorporated to calculate the eye-camera magnification to provide an estimate of the true or absolute measurements of retinal structures (e.g., the optic nerve) on the fundus photograph.

The location of the intermediate real image of the fundus as created by the front lens of the camera depends on the optical power of the eye being photographed. The setting or position of the focusing mechanism on the camera to best see the intermediate image is indicative of the glass refractive error of the eye 2520. This measurement of the glass refraction of the eye 2520 may be used to calculate the eye-camera magnification factor produced on a fundus image.

For example, if the size of the optic disc is desired, the eye-camera magnification factor is used with the area of the disc occupied in the image (e.g., the area can be measured in pixels using image processing software such as Adobe® Photoshop® software) to arrive at an area measurement of the disc. This area measurement can then be corrected to yield an approximation of the true disc area with “correction factors” (see, e.g., A. R. Rudnicka et al., “Magnification Characteristics of Fundus Imaging Systems,” Ophthalmology, 105(12):2186-92 (December 1995)) or deriving “correction factors” from a standard group of subjects whose disc area has been determined by techniques as described above.

In experiments and tests performed, it has been found that the relation between focus mechanism position and glass refraction is highly correlated. Two telecentric fundus cameras, the Topcon TRC-50F50FT and the Topcon TRC-50x, were used to perform twenty degree, red free photographs of the optic nerve in twenty subjects (N=11 with the Topcon TRC-50F50FT and N=9 with the Topcon TRC-50x). A vernier scale was attached to the focusing knob which permitted a measurement of the knob position to be recorded. A correlation of this measurement with the eye refractive error was performed. The position of the focusing knob on both cameras correlated highly with the refractive error of the eye being photographed r=0.97 for the TRC-50F50FT and r=0.99 with the TRC50X. It is noted that the photographs are taken in such a fashion so as to minimize the effect that the photographer's own lens accommodation may have on the focusing of the image. This can be done by having a photographer being of sufficient age so that the photographer's own diminished accommodative powers do not interfere with the focusing of the camera, or by ensuring that focusing of the camera is done in such a way as not to employ one's own focusing ability. This problem is reduced or eliminated in cameras that employ an automatic or semi-automatic electronic focusing mechanism.

The position of the focusing mechanism 2503 reflects the optical refractive error of the eye 2520 being photographed which, in turn, is used to calculate the eye-camera magnification. This position measurement can be incorporated into a calculation of the optic disc or other retinal object size. Although the above examples were performed with telecentric cameras, some embodiments employ non-telecentric cameras. Some changes in this technique for retinal object size determination can permit magnification factors for non-telecentric cameras to be calculated. See, e.g., A. R. Rudnicka et al., “Magnification Characteristics of Fundus Imaging Systems,” Ophthalmology, 105(12):2186-92 (December 1995).

Some embodiments provide that the measurement of fundus (retinal) objects from a photographic image captured with a fundus camera is performed by calculating (e.g., manually or automatically) the eye-camera magnification with the formulas that use the length of the eye, the glasses strength of the eye, or the glasses strength and corneal curvature as mentioned above. The known value of the glasses strength of the eye can also be used to set the position of the focusing knob to bring the image of the retina into clear focus when taking the photograph of the retina, without adverse effects resulting from the photographer's own accommodation (see, e.g., Bengtsson and Krakau (1997), p. 131).

According to some embodiments, the position of the focusing mechanism 2503 on the retinal camera is used to calculate the eye-camera magnification. This magnification factor can, in turn, be used along with a measuring tool (e.g., a software measuring tool) whose scale changes according to the eye-camera magnification to calculate retinal object size (e.g., the optic nerve and the optic cup which are indices for diagnosing glaucoma, vessel caliber, or tumor diameter). This is useful for rapid screening of large numbers of people especially with automation of photograph reading. As will be appreciated by one of ordinary skill in the art, the methods and systems according to various embodiments are disclosed in at least the elements (e.g., components, hardware, software, firmware, systems, devices, etc.) and steps (e.g., in any order) as described and/or shown in the drawings herein.

The patient's eye 2520 is positioned for a retinal photograph (or a photograph of the optic nerve). The aerial image of the object is focused on the film or CCD (image plane) with the focusing mechanism 2503. The position of the focusing mechanism 2503 may be recorded with the image. This can also be done by automatic means, such as a position sensor measuring the position of the focusing mechanism 2503 and a digital signal can be obtained for providing a focusing position measurement. The recorded position is then calibrated to the eye optical error (e.g., glass refraction) and is used to calculate the eye-camera magnification. An accurate scale of the fundus image can be determined using the eye-camera magnification calculated in the previous step.

When measuring objects found in the image using a software image analysis tool, the scale determined in the previous step is used to measure linear objects (e.g. vessel widths) or two-dimensional objects (e.g., the optic nerve head) in absolute units (e.g. mm, mm², etc.). The position of the focusing mechanism recorded previously can be used in future photographs of the same patient to help control for inter-session eye-camera magnification variability by helping place the camera in front of the eye.

However, once the diagnosis of an ocular condition necessitating a fundus photograph is made (e.g., glaucoma) in a given patient, the eye-camera magnification may be constant (unless, of course, the patient has had surgery to correct for myopia or cataract surgery or develops a condition such as a cataract which could change the glass refraction of the eye). As mentioned, the position of the focusing mechanism on the camera can be used to help minimize variability of magnification between photo sessions. For each session, the patient sits in front of the camera and the camera is manually moved to a fixed distance (e.g., 10 cm) from the patient which is determined by the photographer. There is a certain error in this positioning since it is manually done. This error can cause a change in the position of the focusing mechanism and thus a change in eye-camera magnification. Knowing and consistently using the position of the focusing mechanism of the previous photographic session can decrease image magnification variability over multiple sessions.

Examples and Refractive Measurements

To demonstrate that the refractive errors of the patients can be extracted from different retinal fundus imaging systems there are a few examples demonstrated below, including Zeiss Oberkochen WS240; Nikon NF505; SLO prototype (UK); LTS (Heidelberg); and HRT.

Ocular retraction (in red) was recorded in the different retinal imaging systems and demonstrated below in Tables 1 and 2.

TABLE 1
Refractive Errors registered/stored in different retinal cameras

| | | | 95% Confidence |
|---|---|---|---|
| | | Range of Ocular | Interval for Repeated |
| | | Refraction | Measurements |
| | Instrument | Investigated (D) | of s (%) |
| Example-1 | Zeiss Oberkochen | +10 to −11 | +2.30/−2.54 |
| | WS240 | | |
| Example-2 | Nikon NF505 | +15.3 to −11.5 | +3.03/−2.67 |
| | 20° field | | |
| Example-3 | SLO prototype (UK) | +15.5 to −11.6 | +3.12/−2.88 |
| Example-4 | LTS (Heidelberg) | +11 to −7 | +2.98/−3.08 |
| | 10° field | | |
| Example-5 | HRT (10° field) | +11 to −11 | +2.01/−1.85 |

SLO = scanning laser ophthalmoscope; LTS = laser tomographic scanner; HRT = Heidelberg Retina Tomography.

TABLE 2
Linear Relationship between P and Ocular Refraction K

| | | | 95% Confidence |
|---|---|---|---|
| | | Range of Ocular | Interval for Repeated |
| | | Refraction | Measurements |
| Instrument | | Investigated (D) | of s (%) |
| Example -1 | | | |
| Canon | | +17.5 to −14 | +2.92/−3.00 |
| CR4-45NM | | | |
| (UK) | | | |
| Example -2 | | | |
| Canon CF60S | | +12 to −8 | +2.01/−2.28 |
| (Cologne) | | | |
| Normal camera | | | |
| setting | | | |
| Example -3 | | | |
| Nidek 3-DX | 30° field | +19 to −8 | +2.13/−2.95 |
| Example -4 | | | |
| Carl Zeiss | 60° field | +19 to −8 | |
| Jena | | | |
| Retinophot | | | |
| Example -5 | | | |
| Canon 60U | | +21 to −8 | +3.03/−2.55 |
| Example -6 | | | |
| Olympus GRCW | 18° field | +12 to −12 | +3.04/−4.65 |
| | 30° field | +11 to −12 | +2.77/−3.02 |

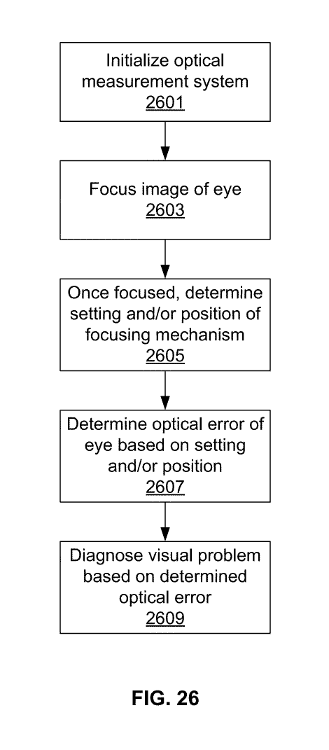

FIG. 26 shows a flowchart illustrating an embodiment of a method for diagnosing a visual problem. Referring to FIG. 26, the example steps begin with step 2601 where the optical measurement system is initialized, which may include activating a sensor or sensors, a focusing mechanism, and processing capability and placing a patient in the measurement system to allow focusing on the patient's eye. In step 2603, the optical measurement system may be focused on the eye, followed by step 2506 where the setting and/or position of the focusing mechanism may be determined.

In step 2607, the optical error of the eye may be determined based on the setting and/or position of the focusing mechanism using techniques described with respect to FIGS. 1-25. Finally, in step 2609, visual problems of the patient's eye may be diagnosed based on the determined optical error.

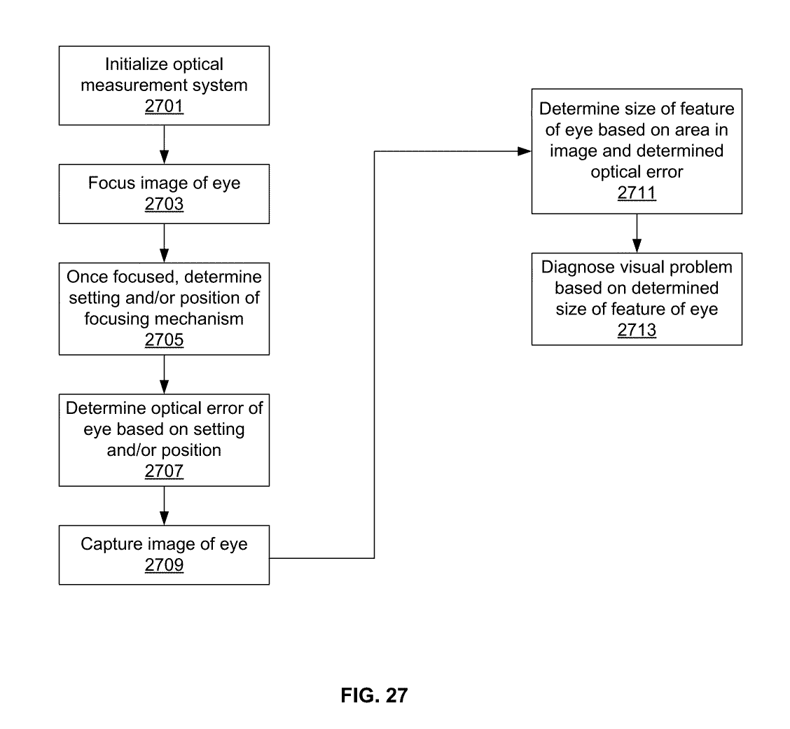

FIG. 27 shows a flowchart illustrating an embodiment of a method for determining a size or a length of a feature of an eye in absolute units. Referring to FIG. 27, the example steps begin with step 2701 where the optical measurement system is initialized, which may include activating a sensor or sensors, a focusing mechanism, and processing capability and placing a patient in the measurement system to allow focusing on the patient's eye. In step 2703, the optical measurement system may be focused on the eye, followed by step 2706 where the setting and/or position of the focusing mechanism may be determined.

In step 2707, the optical error of the eye may be determined based on the setting and/or position of the focusing mechanism using techniques described with respect to FIGS. 1-25. In step 2709, an image of the patients eye may be captured and in step 2711, a size of a feature of the patient's eye may be determined based on the area in the image and the optical error. Finally, in step 2713, visual problems of the patient's eye may be diagnosed based on the determined optical error and the size of the feature.

FIG. 28 shows a block diagram of some components of an embodiment of an imaging system or imaging subsystem. Referring to FIG. 28, there is shown an optical measurement system 2800 comprising one or more processors 2801, a memory 2803, a communication interface 2805, one or more sensors 2807, a focusing mechanism 2809, input/output components 2811, a bus 2813, and optical components 2815.

The one or more processors 2801 may comprise a general purpose processor, for example, that may be operable to control the operations of the optical measurement system 2800. The memory 2803 may comprise a non-transitory storage medium, or a combination of transitory and non-transitory storage medium, that may be operable to store operational settings of the optical measurement system 2800 as well as output data and images from the sensors 2807.

The communication interface 2805 may comprise a wired and/or wireless interface for communication with external devices, such as a computer system, for example. The communication interface 2805 may be operable to communicate over one or more wired and/or wireless standards such as USB, IEEE 801.11x, Bluetooth, etc.

The sensors 2807 may comprise one or more semiconductor die that comprise an array of CMOS or CCD sensing devices that may be operable to sense visible and/or infrared light, depending on the light source and the wavelength sensitivity of the eye structure to be measured. The sensors 2807 may generate data that may be processed by the processor 2801 and stored in the memory 2803.

The focusing mechanism 2809 may comprise electrical and/or electro-mechanical elements in the optical measurement system 2800 that configures the position of one or more lenses such that a target object or structure is in focus. The focusing mechanism 2809 may comprise a focusing knob, either mechanical or electronic. In another example scenario, the focusing mechanism 2809 may comprise a Vernier scale to enable repeated measurements with similar settings.

The input/output components 2811 may comprise devices for displaying output or for inputting data to the optical measurement system 2800, and may comprise displays, printers, keyboards, mouse, storage, etc. The bus 2813 may comprise an electrical interconnection between each of the electronic components of the optical measurement system 2811 such that each may communicate with the others. For example, the processor may cause data from the sensors 2807 to be stored in the memory 2803 via the bus 2813.

The optical components 2815 may comprise lenses for capturing optical signals. The distance between lenses may be configured by the focusing mechanism 2809.

In operation, the optical measurement system 2800 may be activated or powered up and a patient may be situated in a position that the patient's eye may be imaged. The focusing mechanism 2809 may be utilized to configure the position of the optical components 2815 such that the patient's fundus or other structure is in focus. The processor 2801 may determine the setting and/or position of the focusing mechanism 2809 where the fundus or other structure is in focus.

The processor 2801, or an external processor if data is communicated via the communication interface, may determine the optical error of the eye based on the determined setting and/or position. In addition, an image may be taken of the subject utilizing the sensors 2807 and a size of one or more features in the captured image may be determined utilizing the processor 2801 or an external processor, for example. In addition, visual problems of the patient's eye may be diagnosed based on the determined optical error and the size of the feature.

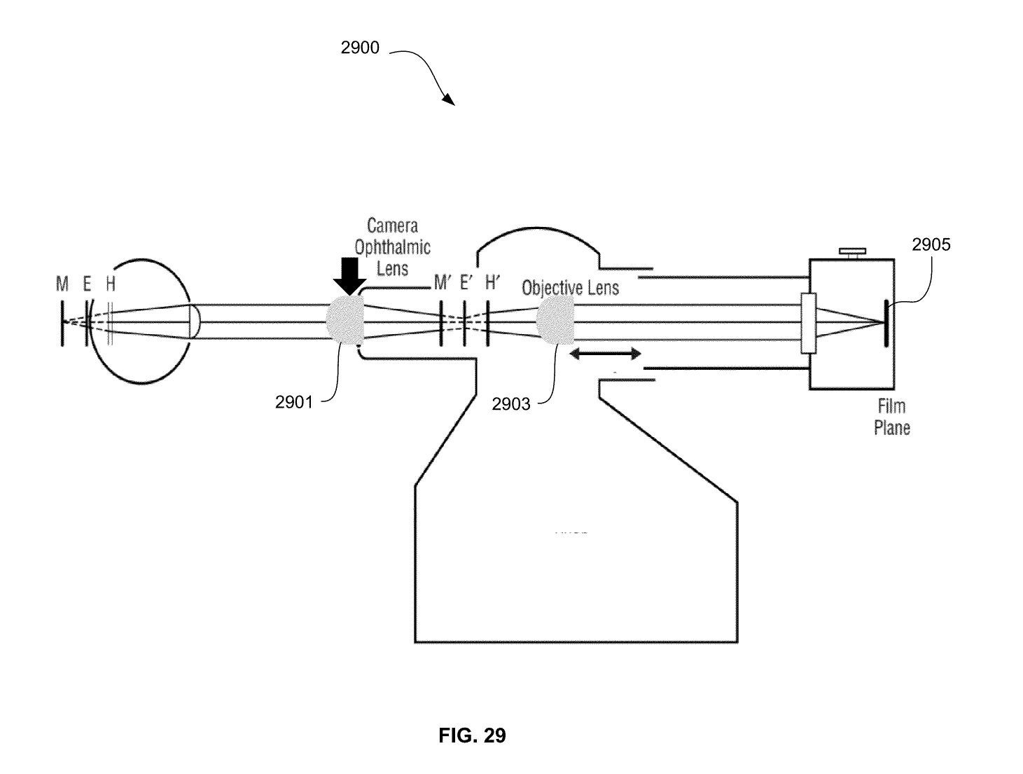

FIG. 29 shows a retinal camera refraction. Referring to FIG. 29, there is shown a retinal camera 2900 imaging an eye, where the luminesced retinal structures are projected through an ophthalmoscopic lens 2901 of the camera and form an image (the first aerial image of the fundus). Myopic (M), Emmetropic (E), and Hyperopic (H) retinal images form their respective aerial images (M′, E′, and H′) inside the camera 2900. The distance between the camera objective lens 2903 and the film plane 2905 is altered with the focusing mechanisms to bring the desired image into focus on the film plane 2905.

A typical camera is equipped with a manual or automatic focusing mechanism to compensate for the refractive errors in the eye. The location of the intermediate real image of the fundus as created by the front lens of the camera depends on the optical power of the eye being photographed. The setting or position of the focusing mechanism on the camera to best see the intermediate image therefore reflects the spectacle refractive error of the eye.

The optical design of the fundus camera and scanning system is similar to that of the indirect ophthalmoscope. A light source (white light or laser) within the camera or scanning laser ophthalmoscopic system illuminates the retina. The luminous retinal structures are projected through the pupil and then through the front lens or ophthalmoscopic lens of the camera/scanning system where an image is formed.

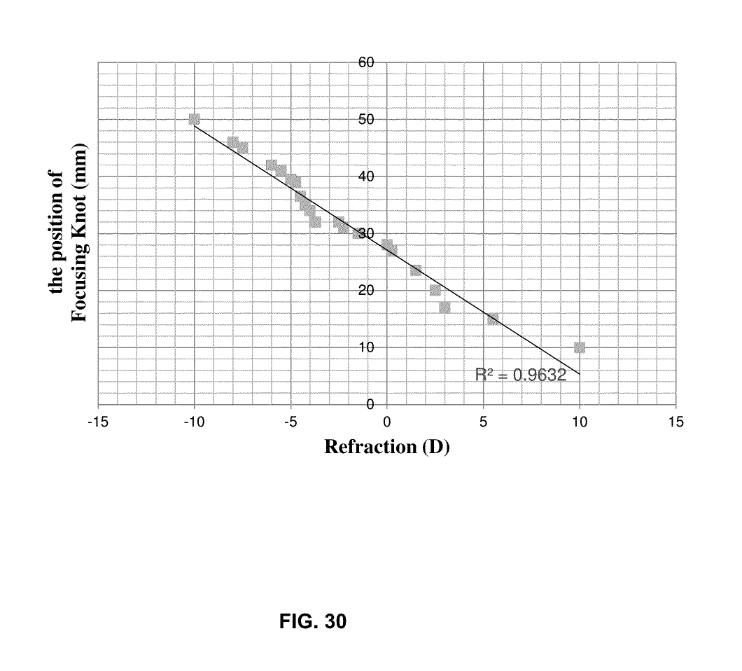

Accumulated studies have discovered that there is a linear relationship between focusing knob position and the change in spectacle refraction. It has been found that the distance between the camera objective and the film plane or the position of this focusing knob reflects the spectacle refraction of the eye examined. In other words, once the camera is appropriately positioned and focused on the retina, the position between the camera objective lens and the film plane reflects the absolute spherical equivalent refraction of the subject being photographed, as illustrated in FIG. 30.

This is mainly because the ophthalmoscopic lens of any given camera/scanning system is usually a constant, and the distance from the eye being photographed is fixed, the position of the first aerial image in relation to the lens can be therefore seen as a function of the optical error or spectacle refraction of the eye. This first aerial image is then brought into focus on the film plane by means of the focusing mechanism (often a focusing knob located on the side of the photographic system or one or more automatic focusing systems, which are attached to the camera body) to adjust the distance between the camera objective and the film plane.

FIG. 30 illustrates a linear regression curve of spectacle refraction and focusing position. In the plot, a linear regression curve of the a Topcon TRC-50X camera with spectacle refraction in diopters (D) is shown as an example. The spectacle refraction (D) on the x-axis is plotted as function of focusing knob position (on the y-axis) where the coefficient of determination, R², is 0.9632 and N=19. Therefore, the focusing knob position provides a good measure of spectacle refraction.

Since the final focusing position of the camera/the scanning system is correlated highly with each patient's spectacle refraction as shown in FIG. 30, the refraction of the eye being photographed can therefore be collected or by incorporating computing software or an optometry to the camera or scanning system.

For example, it has been found that the relationship between focus mechanism position and spectacle refraction is highly correlated. Telecentric fundus cameras—the Topcon TRC-50F50FT and the Topcon TRC-50x were used to perform twenty degree red free photographs of the optic nerve in twenty subjects (N=1 1 with the Topcon TRC-50F50FT and N=9 with the Topcon TRC-50x). A vernier scale was attached to the focusing knob which permitted a further refinement of the measurement. A correlation of this measurement with the eye refractive error was performed. The position of the focusing knob on both cameras correlated highly with the refractive error of the eye being photographed r=0.97 for the TRC-50F50FT and r=0.99 with the TRC50X.

These discoveries described are in agreement with the optometer principle that, in brief, permits continuous linear variation of power in refracting instruments, including automatic refractometers and lensometers In this way, the fundus camera and scanning system can be regarded as a refracting instrument. The linear relationship has been used as a means to help photographers with flexible accommodation to obtain better retinal photographs.

Automated Refraction

Autorefractors comprise an infrared source, a fixation target and a Badal optometer. An infrared light source (around 800-900 nm) is used primarily because of the ocular transmission and reflectance characteristics achieved at the sclera. At this wavelength, light is reflected back from the deeper layers of the eye (choroid and sclera) and this, together with the effects of longitudinal chromatic aberration, means that a systematic error of approximately −0.50 DS may be added to compensate for ocular refraction with visible light. A variety of targets have been used for fixation ranging from less interesting ‘stars’ to pictures with peripheral blur to further relax accommodation. Autorefractors now use the fogging technique to relax accommodation prior to objective refraction. Practitioners may recall in the past patients stating that the target is blurred prior to measurements being taken—this is the effect of the fogging lens. However, even with this fogging technique, micro fluctuations in accommodation occur up to 0.50 DS5. Some of this effect is counteracted by averaging multiple readings—however, the error is not eliminated.

Autorefractors typically comprise a Badal optometer within the measuring head. The Badal lens system has two main advantages. Firstly, there is a linear relationship between the distance of the Badal lens to the eye and the ocular refraction within the meridian being measured. Secondly, with a Badal lens system, the magnification of the target remains constant irrespective of the position of the Badal lens.

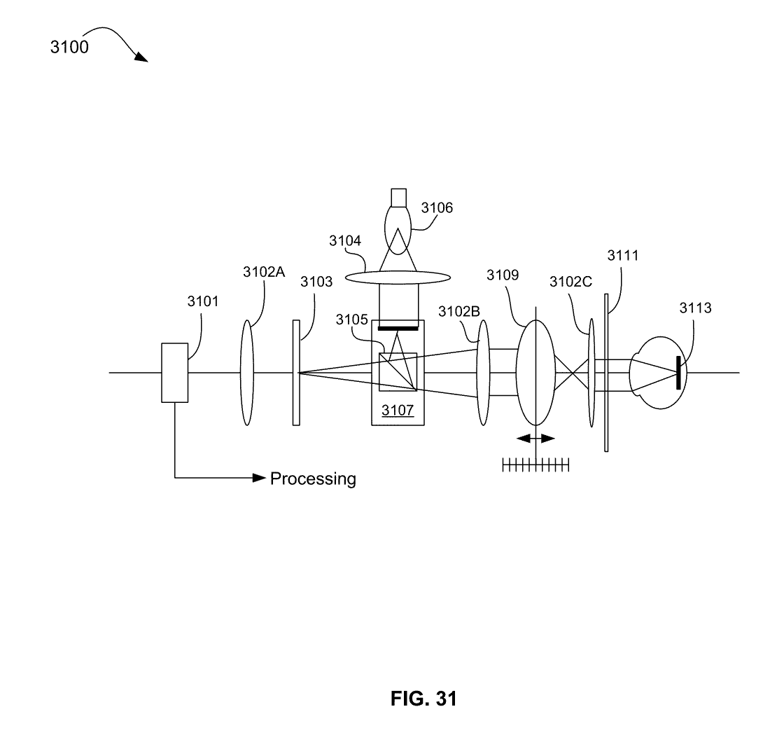

FIG. 31 illustrates the basic principle of the autorefractor. Infrared light is collimated by a condensing lens 3104 and passes through rectangular masks housed in the rotating chopper 3107. The light passes through a beam splitter 3105 to the optometer system. This system moves laterally, as indicated by the scale below the moving lens 3109, to find the optimal focus of the slit of the slit mask 3103 on the retina. Optimal focus is achieved when a peak signal is received from the light sensor 3101.

The polarizing beam splitter 3105 effectively removes reflected light from the cornea whereas the slit image 3113 on the retina passes through the polarized beam splitter 3105. The system may measure at least three meridians of the eye in order to derive the refractive power of the eye using the sine-squared function.

The sine-squared function of ocular astigmatism describes the variation of meridional astigmatic power. Thus, for any given prescription sph/−cylxθ, the power along any given meridian is given by the formula sph+(cyl×sine2θ). Autorefractors only need to calculate the power at three chosen meridians in order to calculate the sphero-cylindrical prescription using the sine-squared function. Basically, the three power measurements at the three respective meridians provide three points on the sine-squared function graph. From this, the rest of the curve can be extrapolated in order to calculate the maximum and minimum power values, i.e. the principal focal planes.

There are three types of autorefractors which derive objective refraction by: 1) Image quality analysis, 2) Scheiner double pin-hole refraction; and 3) Retinoscopy.

Image Quality Analysis

This method is not in common use today in autorefractors. It was originally used in the Dioptron autorefractor. The optimal position of the Badal optometer lens was determined by the output signal of the light sensor. The rotating drum with an opening produces a light/dark alternating target and the light sensor matches the intensity profile of the incoming light from the eye, to the light intensity pattern from the rotating slit drum. A low intensity profile indicates that the Badal lens is not in the correct position to correct the meridional power, and at a peak intensity profile, the Badal optometer reading is taken to signify the power of the meridian being measured. Once this is performed for three meridians, the sine-squared function may be used to derive the sphero-cylindrical prescription. A lens prescription typically includes both a sphere (or spherical) component and a cylinder (or cylindrical) component, which is known as a spherocylinder (or spherocylindrical) prescription.

Scheiner Double Pin-Hole Refraction

Most autorefractors used in practice today use the Scheiner principle. In a clinical setting, a double pin-hole identifies the level of ametropia in a subject by placing it directly in front of the patient's pupil. In a myopic eye, the patient sees crossed diplopic images, whereas in hyperopia, the patient sees uncrossed images. Crossed and uncrossed doubling can easily be differentiated by asking the patient which image has disappeared, when either top or bottom pin-hole is occluded. Implementation of this technology in autorefractors is somewhat different. In general, two LEDs (light emitting diodes) may be used as the infrared source and imaged to the pupillary plane. These effectively act as a modified Scheiner pinhole by virtue of the narrow pencils of light produced by the small aperture pinhole located at the focal point of the objective lens.

Once the LEDs are imaged in the pupillary plane, ocular refraction leads to doubling of the LEDs if refractive error is present. After refraction, the retinal image of the LEDs reflects from the retina back out of the eye. However, light emanating from the eye is again reflected by a semi-silvered mirror to a dual photodetector. In order to differentiate between crossed and uncrossed doubling, the LEDs flicker alternately at a high frequency. The dual photodetector image may be designed to image only one of the two LEDs in each half. As a result, crossed and uncrossed diplopia can be detected. As the LED system is moved back and forth (according to the type of diplopia), the separation of the diplopic images varies on the photodetector. When the retinal image is single, a single LED image is centered over both photodetectors. The LED position corresponds to the refractive error in that meridian. In the case of astigmatism, four LEDs are used and the power perpendicular to the meridian under test is measured.

Retinoscopy Based

Some autorefractors use infra-red videorefraction where a grating, or slit, is produced by a rotating drum. Similar principles to retinoscopy are used where the speed of the reflex is used as an indicator of the patient's refraction. The optical configuration was originally described by Foucault and was used to test the surface quality of mirrors. It is now better known as the ‘knife test’ where the slit (or ‘knife’ as it was originally called) was produced using a pair of blades side by side. The slit is used to determine the refractive power of the eye. The speed and direction of the movement of the reflex is detected by photodetectors and computed to derive the meridional power.

The vertical slit calculates the refraction of the vertical meridian. The system detects that the vertical meridian is measured by the way each detector senses the slit as it passes over the pupil. The time difference from the slit reaching each of the detectors allows the autorefractor to detect the meridian under investigation. The oblique slit will likewise initiate a different time dependent response from the detectors, and thus derive the power within the oblique meridian. Once the optimum movement is derived corresponding to neutralization in that meridian, the dioptric value is plotted on a sine-squared function to derive the sphero-cylindrical refraction.

A method and system for visual problem diagnosis using refractive parameters measured with a retinal camera may comprise focusing, using a focusing mechanism, an image of a fundus, determining a setting or a position of the focusing mechanism, and determining an optical error of an eye based on the determined setting or position of the focusing mechanism. The focusing mechanism may comprise a focusing knob. The determined setting or position of the focusing mechanism may be indicative of a refractive error of the eye. The determined setting or position of the focusing mechanism may be indicative of a glass refractive error of the eye. The focusing mechanism may be part of a fundus camera.

The focusing mechanism may be part of an indirect ophthalmoscopic device. The optical error may comprise a refraction of the eye which is based on an object-to-image size ratio or magnification. The image may be an intermediate real image of the fundus. A location of the intermediate real image of the fundus may be determined which is indicative of an optical power of the eye. An image of the fundus may be captured and a size of the fundus may be determined based on the captured image and the determined optical error. An image of a feature of the eye may be captured and a size of the feature of the eye may be determined based on a determined area of the feature occupied in the captured image and the determined optical error.

The determined optical error may be a refraction of the eye which is used to calculate a magnification factor. The focusing mechanism may include or be coupled to a Vernier scale, wherein the Vernier scale is used to determine the position of the focusing mechanism. The determined optical error of the eye may be correlated with the determined position of the focusing mechanism. The determined optical error may be used to calculate an eye-camera magnification. The method may be performed by a fundus camera or a telecentric camera.

Some embodiments according to the present disclosure may be realized in hardware, software, firmware or a combination of hardware, software or firmware. Some embodiments according to the present disclosure may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

Some embodiments according to the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: (a) conversion to another language, code or notation; and (b) reproduction in a different material form.

While some embodiments according to the present disclosure have been described with reference to certain embodiments, it will be understood by those skilled in the art that various changes may be made and equivalents may be substituted without departing from the scope of the present disclosure. In addition, the present disclosure contemplates that aspects and/or elements from different embodiments may be combined into yet other embodiments according to the present disclosure. Moreover, many modifications may be made to adapt a particular situation or material to the teachings of the present disclosure without departing from its scope. Therefore, it is intended that the present disclosure not be limited to the particular embodiments disclosed, but that the present disclosure will include all embodiments falling within the scope of the appended claims.

What is claimed is:

1. A method for diagnosing visual problems, comprising:
focusing, using a focusing mechanism in an autorefractor, an image of a fundus;
determining a setting or a position of the focusing mechanism; and
determining an optical error of an eye based on the determined setting or position of the focusing mechanism.

2. The method according to claim 1, wherein the focusing mechanism comprises a focusing knob.

3. The method according to claim 1, wherein the determined setting or position of the focusing mechanism is indicative of a refractive error of the eye.

4. The method according to claim 1, wherein the determined setting or position of the focusing mechanism is indicative of a glass refractive error of the eye.

5. The method according to claim 1, wherein the focusing mechanism is part of a fundus camera.

6. The method according to claim 1, wherein the focusing mechanism is part of an indirect ophthalmoscopic device.

7. The method according to claim 1, wherein the optical error comprises a refraction of the eye which is based on an object-to-image size ratio or magnification.

8. The method according to claim 1, wherein the image is an intermediate real image of the fundus.

9. The method according to claim 9, comprising:
determining a location of the intermediate real image of the fundus which is indicative of an optical power of the eye.

10. The method according to claim 1, comprising:
capturing an image of the fundus; and
determining a size of the fundus based on the captured image and the determined optical error.

11. The method according to claim 1, comprising:
capturing an image of a feature of the eye; and
determining a size of the feature of the eye based on a determined area of the feature occupied in the captured image and the determined optical error.

12. The method according to claim 11, wherein the determined optical error is a refraction of the eye which is used to calculate a magnification factor.

13. The method according to claim 1, wherein the focusing mechanism includes or is coupled to a Vernier scale, wherein the Vernier scale is used to determine the position of the focusing mechanism.

14. The method according to claim 1, wherein the determined optical error of the eye is correlated with the determined position of the focusing mechanism.

15. The method according to claim 1, wherein the determined optical error is used to calculate an eye-camera magnification.

16. The method according to claim 1, wherein the method is performed by a fundus camera or a telecentric camera.

17. A camera system, comprising:
one or more processors;
one or more non-transitory memories coupled to the one or more processors;
one or more lenses;
one or more focusing mechanisms configured to control the one or more lenses
wherein processor-executable instructions or code are stored in the one or more processors or the one or more non-transitory memories, wherein the execution of the processor-executable instructions or code by the one or more processors causes the one or more processors to perform the following:
determine a setting or a position of the focusing mechanism when the intermediate real image of the fundus is focused, and
determine a refraction of an eye being viewed by the camera system based on the determined setting or position of the focusing mechanism.

18. The camera system according to claim 17, comprising:
sensors operatively coupled to the one or more processors, wherein the sensors are configured to capture an image of the fundus of the eye.

19. The camera system according to claim 17, wherein the execution of the processor-executable instructions or code by the one or more processors causes the one or more processors to perform the following:
determine an area of the fundus occupied in the captured image, and
determine a size of the fundus of the eye based on the determined area of the fundus and the determined refraction of the eye.

20. The camera system according to claim 19, wherein the determined refraction of the eye comprises a glass refraction of the eye.

21. The camera system according to claim 18, wherein the sensors comprise one or more CCD arrays.

22. The camera system according to claim 18, wherein an aerial image of the fundus is focused on the sensors.

23. The camera system according to claim 17, wherein the execution of the processor-executable instructions or code by the one or more processors causes the one or more processors to perform the following:
calibrate or correlate the determined refraction of the eye with the determined setting or position of the focusing mechanism.

24. The camera system according to claim 17, wherein the determined setting or position of the focusing mechanism is used for capturing additional images of the fundus of the eye.

25. A camera system, comprising:
one or more lenses in an autorefractor;
one or more focusing mechanisms configured to control the one or more lenses; and
one or more sensors configured to receive light through the one or more lenses from an eye,
wherein the one or more focusing mechanism are configured to focus light from the an eye that is received by the one or more sensors,
wherein a setting or a position of the focusing mechanism is determined, and
wherein an optical error of the eye is determined based on the determined setting or the determined position of the focusing mechanism.

26. The camera system according to claim 25, wherein an image of the eye is captured by the one or more sensors, wherein a size of at least a portion of the eye is determined based on at least the captured image of the eye and determined optical error of the eye.

27. The camera system according to claim 25, wherein the camera comprises a retinal camera or a fundus camera.

28. The camera system according to claim 25, wherein the optical error comprises refractive parameters.

29. The camera system according to claim 25, wherein the one or more sensors comprise one or more CCD arrays.

30. The camera system according to claim 25, wherein the determined optical error provides an indication of a visual problem.

31. The camera system according to claim 25, wherein the optical error comprises a refraction of the eye.

32. The camera system according to claim 25, wherein the optical error comprises a glass refraction of the eye.

33. The camera system according to claim 25, wherein the setting or the position of the focusing mechanism is determined using a vernier scale.

34. A system for diagnosing visual problems, the system comprising:
an autorefractor including a focusing mechanism and a slit mask;
a processor operatively coupled to the autorefractor and a non-transitory storage media,
wherein the non-transitory storage media has stored thereon executable instructions which, when executed by the processor, perform the following:
receiving an image of a fundus from the autorefractor;
configuring a setting or a position of the focusing mechanism thereby moving at least one lens to find an optimal focus of a slit of the slit mask in a retina of an eye; and
determining an optical error of the eye based on the configured setting or the position of the focusing mechanism.
